(19) United States
(12) Patent Application Publication
(10) Pub. No.: US 2006/0116401 A1
(43) Pub. Date: Jun. 1, 2006

(54) Antibacterial compounds

(75) Inventors: Michael Barry Gravestock, Waltham, MA (US); Neil James Hales, Cheshire (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/536,729

(22) PCT Filed: Nov. 24, 2003
(86) PCT No.: PCT/GB03/05082

§ 371 Date: May 27, 2005

(30) Foreign Application Priority Data
Nov. 28, 2002 (GB) 0227701.0

(51) Int. Cl. A61K 31/444; A61K 31/4439; A61K 31/422; C07D 413/14

(52) U.S. Cl. 514/332; 514/340; 514/376; 546/256; 546/271.4; 548/229

(57) ABSTRACT

A compound of the formula (I), or a pharmaceutically-acceptable salt, or in-vivo hydrolysable ester thereof wherein in (I) C is for example formula (D), (E), (H) wherein A and B are independently selected from formulae (i) and (ii) and R₂b and R₆b, R₂b and R₆a, R₃a and R₅a, are for example selected from H, F, OMe and Me; R₂b′ and R₆b′, R₂a′ and R₆a′, R₃a′, R₅a′ are for example selected from H, OMe and Me; R₁a and R₁b are for example selected from hydroxy, —OSi(tri-(1-6C)alkyl), NR₅C(═W) R₄, formla (a), formula (b) wherein HET-1 is for example isoxazolyl and HET-2 is for example triazolyl or tetrazolyl. Methods for making compounds of the formula (I), compositions containing them and their use as antibacterial agents are also described.

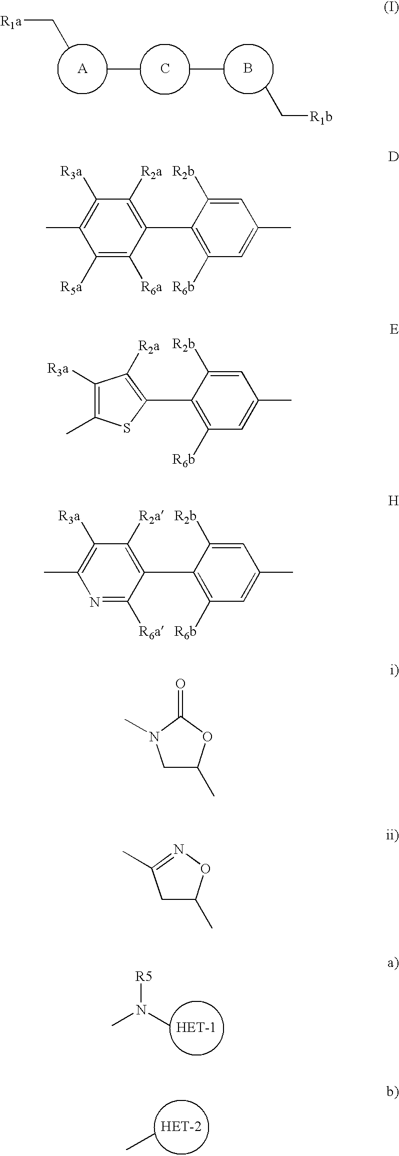

The present invention relates to antibiotic compounds and in particular to antibiotic compounds containing substituted oxazolidinone and/or isoxazoline rings. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus (MRSA), methicillin resistant coagulase negative staphylococci (MRCNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with various toxicities including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Certain antibacterial compounds containing an oxazolidinone ring have been described in the art (for example, Walter A. Gregory et al in J. Med. Chem 1990, 33, 2569-2578 and 1989, 32(8), 1673-81; Chung-Ho Park et al in J. Med. Chem. 1992, 35, 1156-1165). Bacterial resistance to known antibacterial agents may develop, for example, by (i) the evolution of active binding sites in the bacteria rendering a previously active pharmacophore less effective or redundant, and/or (ii) the evolution of means to chemically deactivate a given pharmacophore, and/or (iii) the evolution of efflux pathways. Therefore, there remains an ongoing need to find new antibacterial agents with a favourable pharmacological profile, in particular for compounds containing new, more potent, pharmacophores.

We have discovered a class of bi-aryl antibiotic compounds containing two substituted oxazolidinone and/or isoxazoline rings which has useful activity against Gram-positive pathogens including MRSA and MRCNS and, in particular, against various strains exhibiting resistance to vancomycin and/or linezolid and against E. faecium strains resistant to both aminoglycosides and clinically used β-lactams, but also to fastidious Gram negative strains such as H. influenzae, M. catarrhalis, mycoplasma spp. and chlamydial strains. The compounds of the invention contain two groups capable of acting as pharmacophores. The two groups may independently bind at pharmacophore binding sites where the sites may be similar or different, where the similar or different sites may be occupied simultaneously or not simultaneously within a single organism, or where the relative importance of different binding modes to the similar or different sites may vary between two organisms of different genus. Alternatively one of the groups may bind at a pharmacophore binding site whilst the other group fulfills a different role in the mechanism of action.

Accordingly the present invention provides a compound of the formula (I), or a pharmaceutically-acceptable salt, or an in-vivo-hydrolysable ester thereof, 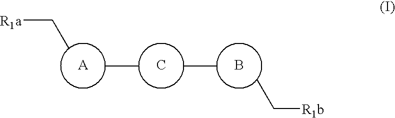
wherein in (I) C is a biaryl group C′-C″ 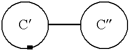
where C′ and C″ are independently aryl or heteroaryl rings such that the group C is represented by any one of the groups D to O below: 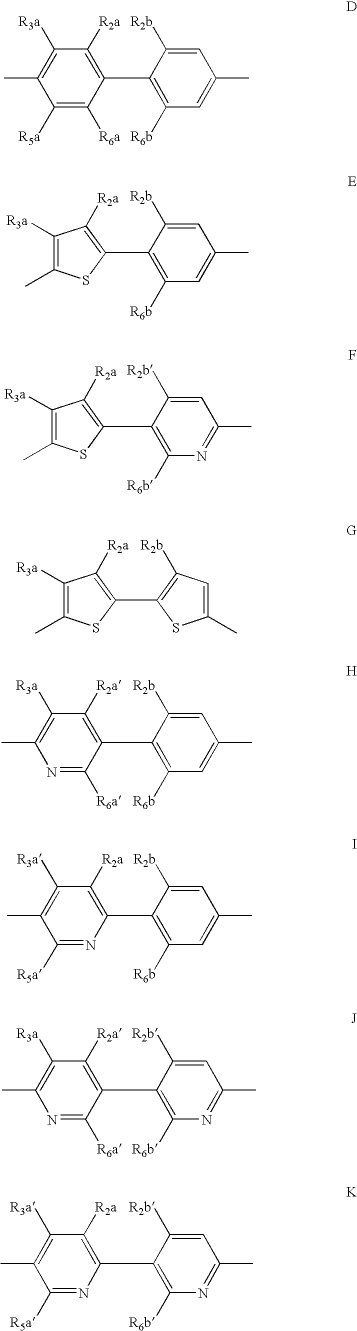 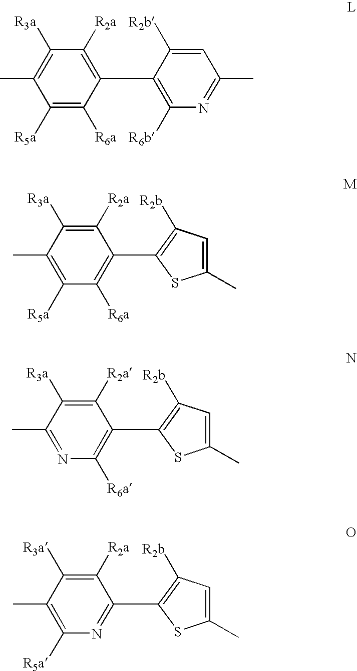
wherein the groups D to O are attached to rings A and B orientation [(A-C′) and (C″-B)] shown and
wherein A and B are independently selected from 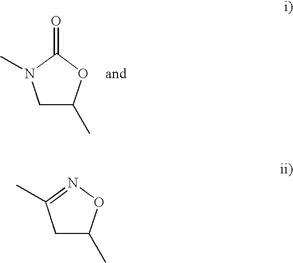
wherein i) and/or ii) are linked as shown in (I) via the 3-position to group C and substituted in the 5-position as shown in (I) by —CH₂—R₁a and —H₂—R₁b;
R₂b and R₆b are independently selected from H, P, Cl, OMe, Me, Et and CF₃;
R₂b′ and R₆b′ are independently selected from H, OMe, Me, Et and CF₃;
R₂a and R₆a are independently selected from H, Br; P, Cl, OMe, SMe; Me, Et and CF₃;
R₂a′ and R₆a′ are independently selected from H, OMe, SMe; Me, Et and CF₃;
R₃a and R₅a are independently selected from H, (1-4C)alkyl, Br, F, Cl, OH, (1-4C)alkoxy, —S(O)ₙ(1-4C)alkyl (wherein n=0, 1, or 2), amino, (1-4C)alkylcarbonylamino, nitro, cyano, —CHO, —CO(1-4C) alkyl, —CONH₂ and —CONH(1-4C)alkyl;
R₃a′, R₅a′ are independently selected from H, (1-4C)alkyl, OH, (1-4C)alkoxy, (1-4C)alkylthio, amino, (1-4C)alkylcarbonylamino, nitro, cyano, —CHO, —CO(1-4C)alkyl, —CONH₂ and —CONH(1-4C)alkyl;
wherein any (1-4C)alkyl group may be optionally substituted with F, OH, (1-4C)alkoxy, —S(O)ₙ(1-4C)alkyl (wherein n=0, 1, or 2) or cyano;
wherein at least one of R₂a′, R₆a′, R₃a, R₅a, R₃a′, and R₅a′ is not H;
wherein when ring C′ is a pyridine ring (ie when group C is group H, L J, K, N or O) the ring nitrogen may optionally be oxidised to an N-oxide;
R₁a and R₁b are independently selected from hydroxy, —OSi(tri-(1-6C)alkyl) (wherein the 3 (1-6C)alkyl groups are independently selected from all possible (1-6C)alkyl groups), —NR₅C(═W)R₄, —OC(═O)R₄, 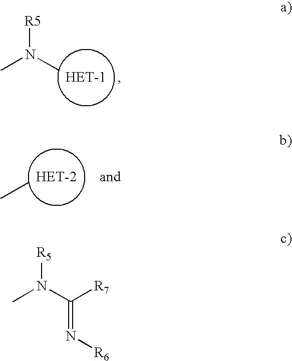
wherein W is O or S;
R₄ is hydrogen, amino, (1-8C)alkyl, —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2; and wherein at each occurrence, alkyl, alkenyl, cycloalkyl cycloalkenyl in substituents in R₄ is optionally substituted with one, two, three or more F, Cl or CN;
R₅ is hydrogen, (3-6C)cycloalkyl, phenyloxycarbonyl, tert-butoxycarbonyl, fluorenyloxycarbonyl, benzyloxycarbonyl, (1-6C)alkyl (optionally substituted by cyano or (1-4C)alkoxycarbonyl), —CO₂R₈, —C(═O)R₈, —C(═O)SR₈, —C(═S)R₈, P(O)(OR₉)(OR₁₀) and —SO₂R₁₁, wherein R₈, R₉, R₁₀ and R₁₁ are as defined hereinbelow;
HET-1 is selected from HET-1A and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom (provided that the ring is not thereby quaternised) by (1-4C)alkyl; HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
HET-2 is selected from HET-2A and HET-2B wherein
HET-2A is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom, which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from RT as hereinafter defined and/or on an available nitrogen atom, other than a N atom adjacent to the linking N atom, (provided that the ring is not thereby quaternised) by (1-4C)alkyl;
HET-2B is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from RT as hereinafter defined and/or on an available nitrogen atom, other than a N atom adjacent to the linking N atom, (provided that the ring is not thereby quaternised) by (1-4C)alkyl;
RT is selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or
(RTa2) (1-4C)alkylamino, di-(1-4C)alkylamino, and (2-4C)alkenylamino;
or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl;
or RT is selected from the group
(RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₆ is cyano, —COR₁₂, —COOR₁₂, —CONHR₁₂, —CON(R₁₂)(R₁₃), —SO₂R₁₂, —SO₂NHR₁₂, —SO₂N(R₁₂)(R₁₃) or NO₂, wherein R₁₂ and R₁₃ are as defined hereinbelow;
R₇ is hydrogen, amino, (1-8C)alkyl, —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2;
R₈ is hydrogen, (3-6C)cycloalkyl, phenyl, benzyl, (1-5C)alkanoyl, (1-6C)alkyl (optionally substituted by substituents independently selected from (1-5C)alkoxycarbonyl, hydroxy, cyano, up to 3 halogen atoms and —NR₁₅R₁₆ (wherein R₁₅ and R₁₆ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₅)(R₁₆) group, R₁₅ and R₁₆ may additionally be taken together with the nitrogen atom to which they are attached to form a pyrrolidinyl, piperidinyl or morpholinyl ring);
R₉ and R₁₀ are independently selected from hydrogen and (1-4C)alkyl;
R₁₁ is (1-4C)alkyl or phenyl;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₂)(R₁₃) group, R₁₂ and R₁₃ may additionally be taken together with the nitrogen atom to which they are attached to form a pyrrolidinyl, piperidinyl or morpholinyl ring which ring may be optionally substituted by a group selected from (1-4C)alkyl, (3-6C)cycloalkyl, (1-4C)alkanoyl, —COO(1-4C)alkyl, —S(O)n(1-4C)alkyl (wherein n=1 or 2), —CS(1-4C)alkyl and —C(═S)O(1-4C)alkyl.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

It will be understood that the phrase “wherein at least one of R₂a′, R₆a′, R₃a, R₅a, R₃a′, and R₅a′ is not H” means that, whichever of said substituents is present in the groups D to O, one of those substituents present must not be hydrogen. For example in group D, R₃a and R₅a are present from the above list of substituents, therefore at least R₃a or R₅a must not be hydrogen. As a further example, in group E, only R₃a is present and therefore this must not be hydrogen. As a further example, in group H, R₂a′, R₆a′ and R₃a are present, so at least one of these must not be hydrogen.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chose from “0, 1 or 2” groups and “1 or 2” groups.

Within this specification composite terms are used to describe groups comprising more that one functionality such as (1-4C)alkoxy-(1-4C)alkoxy-(1-4C)alkyl. Such terms are to be interpreted in accordance with the meaning which is understood by a person skilled in the art for each component part. For example (1-4C)alkoxy-(1-4C)alkoxy-(1-4C)alkyl includes methoxymethoxymethyl, ethoxymethoxypropyl and propxyethoxymethyl.

It will be understood that where a group is defined such that it is optionally substituted by more than one substituent, then substitution is such that chemically stable compounds are formed. For example, a trifluoromethyl group may be allowed but not a trihydroxymethyl group. This convention is applied wherever optional suibstituents are defined.

In this specification, HET-1A and HET-1B are fully unsaturated ring systems.

In this specification, HET-2A may be a fully or partially unsaturated heterocyclic ring, provided there is some degree of unsaturation in the ring.

Particular examples of 5-membered heteroaryl rings containing 2 to 4 heteroatoms independently selected from N, O and S (with no O—O, O—S or S—S bonds) are pyrazole, imidazole, 1,2,3-triazole, 1,2,4-triazole, oxazole, isoxazole, thiazole, 1,2,3-oxadiazole, 1,2,4-oxadiazole, 1,2,5-oxadiazole, 1,3,4-oxadiazole, isothiazole, 1,2,5-thiadiazole, 1,2,4-thiadiazole and 1,2,3-thiadiazole.

Particular examples of 6-membered heteroaryl ring systems containing up to three nitrogen heteroatoms are pyrimidine, pyridazine, pyrazine, 1,2,3-triazine, 1,2,4-triazine and 1,3,5-triazine.

Particular examples of N-linked 5-membered, fully or partially unsaturated heterocyclic rings, containing either (i) 1 to 3 further nitrogen heteroatoms or (ii) a further heteroatom selected from O and S together with an optional further nitrogen heteroatom include, for example, pyrazole, imidazole, 1,2,3-triazole (preferably 1,2,3-triazol-1-yl), 1,2,4-triazole (preferably 1,2,4-triazol-1-yl) and tetrazole (preferably tetrazol-2-yl) and furazan.

Particular examples of N-linked 6-membered di-hydro-heteroaryl rings containing up to three nitrogen heteroatoms in total (including the linking heteroatom) include di-hydro versions of pyrimidine, pyridazine, pyrazine, 1,2,3-triazine, 1,2,4-triazine, 1,3,5-triazine and pyridine.

Particular examples of halogen-substituted alkyl substituents in HET-1 and HET-2 are monofluoromethyl, difluoromethyl and trifluoromethyl.

A particular example of R₈ as a halogen-substituted alkyl group is trifluoromethyl.

In this specification the term ‘alkyl’ includes straight chain and branched structures. For example, (1-4C)alkyl includes propyl and isopropyl. However, references to individual alkyl groups such as “propyl” are specific for the straight chain version only, and references to individual branched chain alkyl groups such as “isopropyl” are specific for the branched chain version only. A similar convention applies to other radicals, for example halo(1-4C)alkyl includes 1-bromoethyl and 2-bromoethyl.

In this specification, the terms ‘alkenyl’ and ‘cycloalkenyl’ include all positional and geometrical isomers.

In this specification, the term ‘aryl’ is an unsubstituted carbocyclic aromatic group, in articular phenyl, 1- and 2-naphthyl.

For the avoidance of doubt, reference to a carbon atom in HBT1 or HET2 being substituted by an oxo or thioxo group means replacement of a CH2 by C═O or C═S respectively.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl and (1-5C)alkyl include methyl, ethyl, propyl, isopropyl and t-butyl; examples of (1-6C)alkyl include methyl, ethyl, propyl, isopropyl, t-butyl, pentyl and hexyl; examples of (1-8C)alkyl include methyl, ethyl, propyl, isopropyl, pentyl, hexyl, heptyl, and octyl; examples of (1-10C)alkyl include methyl, ethyl, propyl, isopropyl, pentyl, hexyl, heptyl, octyl and nonyl; example of —OSi(tri(1-6C)alkyl) are tert-butyldimethylsilyloxy and trimethylsilyloxy; examples of (1-4C)alkanoylamino-(1-4C)alkyl include formamidomethyl, acetamidomethyl and acetamidoethyl; examples of hydroxy(1-4C)alkyl and hydroxy(1-6C)alkyl include hydroxymethyl, 1-hydroxyethyl, 2-hydroxyethyl and 3-hydroxypropyl; examples of (1-4C)alkoxycarbonyl include methoxycarbonyl, ethoxycarbonyl and propoxycarbonyl; examples of (1-5C)alkoxycarbonyl include methoxycarbonyl, ethoxycarbonyl, propoxycarbonyl and butoxycarbonyl; examples of 2-((1-4C)alkoxycarbonyl)ethenyl include 2-(methoxycarbonyl)ethenyl and 2-(ethoxycarbonyl)ethenyl; examples of 2-cyano-2-((1-4C)alkyl)ethenyl include 2-cyano-2-methylethenyl and 2-cyano-2-ethylethenyl; examples of 2-nitro-2-((1-4C)alkyl)ethenyl include 2-nitro-2-methylethenyl and 2-nitro-2-ethylethenyl; examples of 2-((1-4C)alkylaminocarbonyl)ethenyl include 2-(methylaminocarbonyl)ethenyl and 2-(ethylaminocarbonyl)ethenyl; examples of (2-4C)alkenyl include allyl and vinyl; examples of (2-4C)alkenyloxy include allyloxy and vinyloxy; examples of (2-4C)alkenylamino include allylamino and vinylamino; examples of (2-4C)alynyl include ethynyl and 2-propynyl; examples of (1-4C)alkanoyl include formyl acetyl and propionyl; examples of (1-5C)alkanoyl include formyl, acetyl, propionyl and butanoyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkylsulfonyl include methylsulfonyl and ethylsulfonyl; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkoxy-(1-4C)alkoxy-(1-4C)alkoxy include 2-(methoxymethoxy)ethoxy, 2-(2-methoxyethoxy)ethoxy; 3-(2-methoxyethoxy)propoxy and 2-(2-ethoxyethoxy)ethoxy; examples of (1-4C)alkylS(O)₂amino include methylsulfonylamino and ethylsulfonylamino; examples of (1-4C)alkanoylamino and (1-6C)alkanoylamino include formamido, acetamido and propionylamino; examples of (1-4C)alkylcarbonylamino and (1-6C)alkylcarbonyomino include acetamido and propionylamimo; examples of (1-4C)alkoxycarbonylamino include methoxycarbonylamino and ethoxycarbonylamino; examples of N-(1-4C)alkyl-N-(1-6C)alkanoylamino include N-methylacetamido, N-ethylacetamido and N-methylpropionamido; examples of (1-4C)alkylS(O)ₚNH— wherein p is 1 or 2 include methylsulfinylamino, methylsulfonylamino, ethylsulfinylamino and ethylsulfonylamino; examples of (1-4C)alkylS(O)ₚ((1-4C)alkyl)N— wherein p is 1 or 2 include methylsulfinylmethylamino, methylsulfonylmethylamino, 2-(ethylsulfinyl)ethylamino and 2-(ethylsulfonyl)ethylamino; examples of fluoro(1-4C)alkylS(O)ₚNH— wherein p is 1 or 2 include trifluoromethylsulfinylamino and trifluoromethylsulfonylamino; examples of fluoro(1-4C)alkylS(O)ₚ((1-4C)alkyl)NH— wherein p is 1 or 2 include trifluoromethylsulfinylmethylamino and trifluoromethylsulfonylmethylamino examples of (1-4C)alkoxy(hydroxy)phosphoryl include methoxy(hydroxy)phosphoryl and ethoxy(hydroxy)phosphoryl; examples of di-(1-4C)alkoxyphosphoryl include di-methoxyphosphoryl, di-ethoxyphosphoryl and ethoxy(methoxy)phosphoryl; examples of (1-4C)alkylS(O)_{q}— wherein q is 0, 1 or 2 and —S(O)n(1-4C)alkyl wherein n is 0, 1 or 2 include methylthio, ethylthio, methylsulfinyl, ethylsulfinyl, methylsulfonyl and ethylsulfonyl; examples of phenylS(O)_{q} and naphthylS(O)_{q}— wherein q is 0, 1 or 2 are phenylthio, phenylsulfinyl, phenylsulfonyl and naphthylthio, naphthylsulfinyl and naphthylsulfonyl respectively; examples of benzyloxy-(1-4C)alkyl include benzyloxymethyl and benzyloxyethyl; examples of a (3-4C)alkylene chain are trimethylene or tetramethylene; examples of (1-6C)alkoxy-(1-6C)alkyl include methoxymethyl, ethoxymethyl and 2-methoxyethyl; examples of hydroxy-(2-6C)alkoxy include 2-hydroxyethoxy and 3-hydroxypropoxy; examples of (1-4C)alkylamino-(2-6C)alkoxy include 2-methylaminoethoxy and 2-ethylaminoethoxy; examples of di-(1-4C)alkylamino-(2-6C)alkoxy include 2-dimethylaminoethoxy and 2-diethylaminoethoxy; examples of phenyl(1-4C)alkyl include benzyl and phenethyl; example of -(1-8C)alkylaryl include phenyl(1-4C)alkyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of hydroxyimino(1-4C)alkyl include hydroxyiminomethyl 2-(hydroxyimino)ethyl and I-(hydroxyimino)ethyl; examples of (1-4C)alkoxyimino-(1-4C)alkyl include methoxyiminomethyl, ethoxyiminomethyl, 1-(methoxyimino)ethyl and 2-(methoxyimino)ethyl; examples of halo(1-4C)alkyl include, halomethyl, 1-haloethyl, 2-haloethyl, and 3-halopropyl; examples of nitro(1-4C)alkyl include nitromethyl, 1-nitroethyl, 2-nitroethyl and 3-nitropropyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanesulfonamido include methanesulfonamido and ethanesulfonamido; examples of (1-4C)alkylinosulfonyl include methylaminosulfonyl and ethylaminosulfonyl; examples of di-(1-4C)alkylaminosulfonyl include dimethylaminosulfonyl, diethylaminosulfonyl and N-methyl-N-ethylaminosulfonyl; examples of (1-4C)alkanesulfonyloxy include methylsulfonyloxy, ethylsulfonyloxy and propylsulfonyloxy; examples of (1-4C)alkanoyloxy include acetoxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl; examples of (3-8C)cycloalkyl include cyclopropyl, cyclobutyl, cyclopentyl and cyclohexyl; examples of (3-8C)cycloalkenyl include cyclopropenyl, cyclobutenyl, cyclopentenyl and cyclohexenyl; examples of —(CH₂)p(3-8C)cycloalkyl wherein p is 0, 1 or 2 include cyclopropyl, methylcyclopropyl, methylcyclobutyl, methylcyclopentyl and ethylcyclohexyl; examples of —(CH₂)p(3-8C)cycloalkenyl wherein p is 0, 1 or 2 include cyclopropyl, methylcyclopropenyl, methylcyclobutenyl, methylcyclopentenyl and ethylcyclohexenyl; examples of (4-7C)cycloalkyl include cyclobutyl, cyclopentyl and cyclohexyl; examples of di(N-(1-4C)alkyl)aminomethylimino include dimethylaminomethylimino and diethylaminomethylimino.

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 27, 285 (1988); and
e) N. Kakeya, et al., Chem Pharm Bull, 32, 692 (1984).

Suitable pro-drugs for pyridine or triole derivatives include acyloxymethyl pyridinium or triazolium salts eg halides; for example a pro-drug such as: 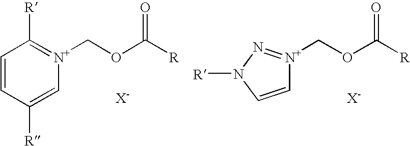
(Ref: T. Yamazaki et al. 42^{nd} Interscience Conference on Antimicrobial Agents and Chemotherapy, San Diego, 2002; Abstract F820).

Suitable pro-drugs of hydroxyl groups are acyl esters of acetal-carbonate esters of formula RCOOC(R,R′)OCO—, where R is (1-4C)alkyl and R′ is (1-4C)alkyl or H. Further suitable prodrugs are carbonate and carabamate esters RCOO— and RNHCOO—.

An in-vivo hydrolysable ester of a compound of the invention or a pharmaceutically-acceptable salt thereof containing a carboxy or hydroxy group is, for example, a pharmaceutically-acceptable ester which is hydrolysed in the human or animal body to produce the parent alcohol

Suitable pharmaceutically-acceptable esters for carboxy include (1-6C)alkoxymethyl esters for example methoxymethyl, (1-6C)alkanoyloxymethyl esters for example pivaloyloxymethyl phthalidyl esters, (3-8C)cycloalkoxycarbonyloxy(1-6C)alkyl esters for example 1-cyclohexylcarbonyloxyethyl; 1,3-dioxolan-2-onylmethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the invention or a pharmaceutically-acceptable salt thereof containing a hydroxy group or groups includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl, carboxy(2-5C)alkylcarbonyl and carboxyacetyl. Examples of ring substituents on phenylacetyl and benzoyl include chloromethyl or aminomethyl, (1-4C)alkylaminomethyl and di-((1-4C)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(1-6C)alkyl-CO— (wherein R^{A} is for example, optionally substituted benzyloxy-(1-4C)alkyl, or optionally substituted phenyl; suitable substituents on a phenyl group in such esters include, for example, 4-(1-4C)piperazino-(1-4C)alkyl, piperazino-(1-4C)alkyl and morpholino-(1-4C)alkyl.

Suitable in-vivo hydrolysable esters of a compound of the formula (I) are described as follows. For example, a 1,2-diol may be cyclised to form a cyclic ester of formula (PD1) or a pyrophosphate of formula (PD2), and a 1,3-diol may be cyclised to form a cyclic ester of the formula (PD3):

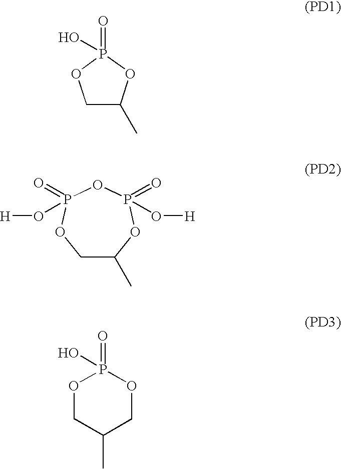

Esters of compounds of formula (I) wherein the HO-function/s in (PD1), (PD2) and (PD3) are protected by (1-4C)alkyl, phenyl or benzyl are useful intermediates for the preparation of such pro-drugs.

Further in-vivo hydrolysable esters include phosphoramidic esters, and also compounds of invention in which any free hydroxy group independently forms a phosphoryl (npd is 1) or phosphiryl (npd is 0) ester of the formula (PD4):

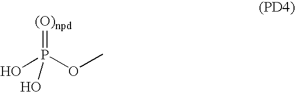

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD4) in which either or both of the —OH groups in (PD4) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), PD2), (PD3) and (PD4) may be prepared by reaction of a compound of invention containing suitable hydroxy group/s with a suitably protected phosphorylating agent (for example, containing a chloro or dialkylamino leaving group), followed by oxidation (if necessary) and deprotection.

Other suitable prodrugs include phosphonooxymethyl ethers and their salts, for example a prodrug of R—OH such as:

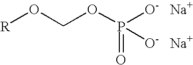

When a compound of invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Where pharmaceutically-acceptable salts of an in-vivo hydrolysable ester may be formed this is achieved by conventional techniques. Thus, for example, compounds containing a group of formula (PD1), (PD2), (PD3) and/or (PD4) may ionise (partially or fully) to form salts with an appropriate number of counter-ions. Thus, by way of example, if an in-vivo hydrolysable ester prodrug of a compound of invention contains two (PD4) groups, there are four HO-P-functionalities present in the overall molecule, each of which may form an appropriate salt (i.e. the overall molecule may form, for example, a mono-, di-, tri- or tetra-sodium salt).

The compounds of the present invention have a chiral centre at both of the C-5 positions of the oxazolidinone and/or isoxazoline rings. The pharmaceutically active diastereomers are of the formula (Ia): 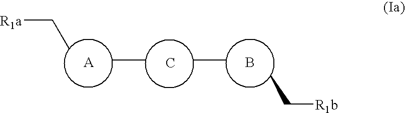
wherein the chiral centre of ring B is fixed in the orientation shown (generally the (5R) configuration, depending on the nature of R₁b, C and B) and ring B is acting as a pharmacophoric group; and wherein the orientation of the chiral centre at ring A may vary and may influence whether ring A also independently binds to a pharmacophore binding site.

The present invention includes pure diastereomers or mixtures of diastereomers, for example a racemic mixture. If a mixture of enantiomers is used, a larger amount (depending upon the ratio of the enantiomers) will be required to achieve the same effect as the same weight of the pharmaceutically active enantiomer.

Furthermore, some compounds of the invention may have other chiral centres. It is to be understood that the invention encompasses all such optical and diastereoisomers, and racemic mixtures, that possess antibacterial activity. It is well known in the art how to prepare optically-active forms (for example by resolution of the racemic form by recrystailisation techniques, by chiral synthesis, by enzymatic resolution, by biotransformation or by chromatographic separation) and how to determine antibacterial activity as described hereinafter.

The invention relates to all tautomeric forms of the compounds of the invention that possess antibacterial activity.

It is also to be understood that certain compounds of the invention can exist in solvated as well as unsolvated forms such as, for example, hydrated forms. It is to be understood that the invention encompasses all such solvated forms which possess antibacterial activity.

It is also to be understood that certain compounds of the invention may exhibit polymorphism, and that the invention encompasses all such forms which possess antibacterial activity.

As stated before, we have discovered a range of compounds that have good activity against a broad range of Gram-positive pathogens including organisms known to be resistant to most commonly used antibiotics, together with activity against fastidious Gram negative pathogens such as H. influenzae, M. catarrhalis, Mycoplasma and Chlamydia strains. The following compounds possess preferred pharmaceutical and/or physical and/or pharmacokinetic properties.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I).

In one aspect, an in-vivo hydrolysable ester of a compound of the formula (I) is a phosphoryl ester (as defined by formula (PD4) with npd as 1).

Compounds of the formula (I), or a pharmaceutically-acceptable salt or an in-vivo hydrolysable ester thereof, wherein group C is represented by any one of groups D to O represent separate and independent aspects of the invention.

Particularly preferred compounds of the invention comprise a compound of the invention, or a pharmaceutically-acceptable salt or an in-vivo hydrolysable ester thereof, wherein the substituents A, B, R₁a, R₁b, R₂a, R₂b, R₃a, R₃b R₅a, R₅a′, R₆a and R₆a′and other substituents mentioned above have values disclosed hereinbefore, or any of the following values (which may be used where appropriate with any of the definitions and embodiments disclosed hereinbefore or hereinafter):

In one embodiment are provided compounds as defined herein in formula (I) or a pharmaceutically-acceptable salt or an in-vivo hydrolysable ester thereof, in which group C is represented by group D.

In another embodiment are provided compounds as defined herein in formula (I) or a pharmaceutically-acceptable salt or an in-vivo hydrolysable ester thereof, in which group C is represented by group E.

In another embodiment are provided compounds as defined herein in formula (I) or a pharmaceutically-acceptable salt or an in-vivo hydrolysable ester thereof, in which group C is represented by group H.

In another embodiment are provided compounds as defined herein in formula (I) or a pharmaceutically-acceptable salt or an in-vivo hydrolysable ester thereof, in which group C is represented by group I.

In another embodiment are provided compounds of formula (I) or a pharmaceutically-acceptable salt or an in-vivo hydrolysable ester thereof, in which the group C is a group represented by any one of groups D, E, H and I as hereinbefore defined.

In a further embodiment are provided compounds of formula (I) or a pharmaceutically-acceptable salt or an in-vivo hydrolysable ester thereof, in which the group C is a group represented by group D or B as hereinbefore defined.

In a further embodiment are provided compounds of formula (I) or a pharmaceutically-acceptable salt or an in-vivo hydrolysable ester thereof, in which the group C is a group represented by group D or H as hereinbefore defined.

In one aspect both A and B are oxazolidinone rings.

In another aspect, either A or B is an oxazolidinone ring and the other is an isoxazoline ring.

In a further aspect, both A and B are isoxazoline rings.

In one aspect, R₂b and R₆b are independently H or R

In one aspect R₂b′ and R₆b′ are both H.

In one embodiment, R₁a and R₁b are independently selected from hydroxy, —NHC(═W)R₄, —OC(═O)R₄, and

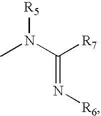

wherein W, R₅ and R₆ are as defined hereinbefore, R₄ is selected from hydrogen, amino, (1-4C)alkyl, —NH(1-4C)alkyl, —N(di-(1-4C)alkyl), —O(1-4C)alkyl, —S(1-4C)alkyl, (2-4C)alkenyl, —(CH₂)p(3-6C)cycloalkyl and —(CH₂)p(3-6C)cycloalkenyl wherein p is 0, 1 or 2; and R₇ is selected from hydrogen, (1-8C)alkyl, —OR₁₂, —SR₁₂, amino, NHR₁₂, N(R₁₂)(R₁₃), (1-8C)alkylaryl and mono-, di-, tri- and per-halo(1-8C)alkyl.

In another embodiment, R₁a and R₁b are independently selected from hydroxy, —NHC(═W)R₄, —OC(═O)R₄, and 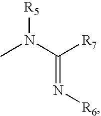
wherein W, R₄, R₅, R₆ and R₇ are as defined hereinbefore, especially wherein R₄ is (1-4C)alkyl, (1-4C)alkoxy, cycloalkyl (particularly cyclopropyl) or haloalkyl (particularly dichloromethyl).

In another embodiment, R₁a and R₁b are independently selected from hydroxy, —NHC(═W)R₄, —OC(═O)R₄, and 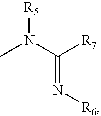
wherein W, R₄, R₅, R₆ and R₇ are as defined hereinbefore, especially wherein R₄ is (1-4C)alkyl or (1-4C)alkoxy.

Particular values for R₅ (which may be used as appropriate with any of the definitions and embodiments disclosed hereinbefore or hereinafter) are hydrogen, tert-butoxycarbonyl and benzyloxycarbonyl. More particularly, R₅ is hydrogen.

When R₁a and/or R₁b is —N(R₅)HET-1, R₅ is preferably hydrogen.

In one aspect R₁₂ and R₁₃ are independently selected from hydrogen, alkyl and aryl, or for any N(R₁₂)(R₁₃) group, R₁₂ and R₁₃ may additionally be taken together with the nitrogen atom to which they are attached to form a pyrrolidinyl, piperidinyl or morpholinyl ring, optionally substituted as hereinbefore described. In one aspect R₁₅ and R₁₆ are independently selected from hydrogen, phenyl and (1-4C)alkyl).

In all of the embodiments, aspects and preferable values for R₁a and R₁b defined hereinbefore or hereinafter, any (1-4C)alkyl group may be optionally substituted as hereinbefore defined. Particular substituents for (1-4C)alkyl groups in definitions for R₁a and R₁b are one or two halogen groups, particularly geminal disubstitution (provided that such substitution is not on a carbon atom attached to an oxygen) and cyano. Examples of di-halosubstituted groups are —NHCOCF₂H and —NHCSCCl₂H.

Preferably R₁a and R₁b are independently selected from hydroxy, —NHCO(1-4C)alkyl, —NHCO(1-4C)cycloalkyl, —NHCS(1-4C)alkyl, —NHCOO(1-4C)alkyl, —NH(C═S)O(1-4C)alkyl, —OCO(1-4C)alkyl, —N(R₅)-HET-1 and HET-2.

More preferably R₁a and R₁b are independently selected from —NHCO(1-4C)alkyl, —NHCO(1-4C)cycloalkyl, —NHCS(1-4C)alkyl, —N(R₅)-HET-1 and HET-2.

In one embodiment R₁a and R₁b are independently selected from hydroxy, —NHCOMe, —NH(C═S)OMe and —NHCOOMe.

In a further embodiment R₁a is selected from hydroxy, —NHCO(1-4C)alkyl (especially —NHCOMe), —NHCO(1-4C)cycloalkyl (especially —NHCOcyclopropyl), —NHCS(1-4C)alkyl (especially —NHCSMe), —NHCOO(1-4C)alkyl (especially —NHCO)Me), —NH(C═S)O(1-4C)alkyl (especially —NH(C═S)OMe) and —OCO(1-4C)alkyl (especially —OCOMe) and R₁b is HET-2.

In a further embodiment R₁a is selected from hydroxy, —NHCO(1-4C)alkyl (especially —NHCOMe), —NHCO(1-4C)cycloalkyl (especially —NHCOcyclopropyl), —NHCS(1-4C)alkyl (especially —NHCSMe), —NHCOO(1-4C)alkyl (especially —NHCO)Me), —NH(C═S)O(1-4C)alkyl (especially —NH(C═S)OMe) and —OCO(1-4C)alkyl (especially —OCOMe) and R₁b is —N(R₅)-HET-1.

In another embodiment R₁a and R₁b are both —NHCO(1-4C)alkyl (especially —NHCOMe) or HET-2 (especially 1,2,3-triazol-1-yl or tetrazol-2-yl).

In a further embodiment R₁a is —NHCO(1-4C)alkyl (especially —NHCOMe) and R₁b is HET-2 (especially 1,2,3-triazol-1-yl or tetrazol-2-yl).

In a further embodiment R₁a is hydroxy and R₁b is selected from —NHCO(1-4C)alkyl (especially —NHCOMe), —NHCO(1-4C)cycloalkyl (especially —NHCOcyclopropyl), —NHCS(1-4C)alkyl (especially —NHCSMe), —NHCOO(1-4C)alkyl (especially-NHCO)Me), —NH(C═S)O(1-4C)alkyl (especially —NH(C═S)OMe) and —OCO(1-4C)alkyl (especially —OCOMe), —N(R₅)-HET-1 (especially where HET-1 is isoxazolyl, 1,2,5-thiadiazolyl or isothiazolyl) and HET-2 (especially 1,2,3-triazol-1-yl or tetrazol-2-yl).

In a further embodiment, R₁a and R₁b are independently selected from hydroxy, acetamido, 1,2,3-triazol-1-yl, methyl-1,2,3-triazol-1-yl and isoxazolylamino.

In a further embodiment, R₁a and R₁b are independently selected from hydroxy, acetamido, 1,2,3-triazol-1-yl, and methyl-1,2,3-triazol-1-yl.

In one embodiment HET-1 and HET-2 are unsubstituted. When substituted, preferred substituents for HET-1 are selected from (1-4C)alkyl, especially methyl, and for HET-2 are selected from halo (particularly chloro), (1-4C)alkyl, especially methyl, mono- and di-halo methyl (wherein halo is preferably fluoro, chloro or bromo), trifluoromethyl and cyanomethyl.

Preferred are HET-1 and HET-2 as 5-membered rings, ie HET-1 as HET-1A and HET-2 as HET-2A, in particular HET-1A as isoxazolyl, 1,2,5-thiadiazolyl or isothiazolyl and HET-2A as 1,2,3-triazol-1-yl or tetrazol-2-yl.

In one aspect, HET-2A as 1,2,3-triazol-1-yl is substituted, preferably by halo (particularly chloro), methyl, difluoromethyl, fluoromethyl, chloromethyl, cyanomethyl or trifluoromethyl.

In one embodiment HET-2A is selected from the structures (Za) to (Zf) below: 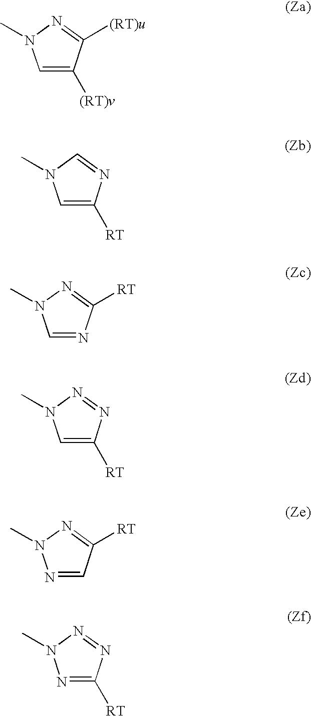
wherein u and v are independently 0 or 1 and RT is as defined in any of the embodiments or aspects defined hereinbefore or hereinafter.

In one embodiment HET-2A is selected from 1,2,3-triazole (especially 1,2,3-triazol-1-yl (Zd)), 1,2,4-triazole (especially 1,2,4-triazol-1-yl (Zc)) and tetrazole (preferably tetrazol-2-yl (Zf)) and wherein u and v are independently 0 or 1 and RT is as defined in any of the embodiments or aspects defined hereinbefore or hereinafter.

In another embodiment HET-2A is selected from 1,2,3-triazol-1-yl (Zd) and tetrazol-2-yl (Zf) and wherein u and v are independently 0 or 1 and RT is as defined in any of the embodiments or aspects defined hereinbefore or hereinafter.

In another embodiment HET-2A is 1,2,3-triazol-1-yl (Zd) and wherein u and v are independently 0 or 1 and RT is as defined in any of the embodiments or aspects defined hereinbefore or hereinafter.

In one embodiment HET-2B is a di-hydro version of pyrimidine, pyridazine, pyrazine, 1,2,3-triazine, 1,2,4-triazine, 1,3,5-triazine and pyridine and wherein RT is as defined in any of the embodiments or aspects defined hereinbefore or hereinafter.

In another embodiment HET-2B is selected from pyrimidone, pyridazinone, pyrazinone, 1,2,3-triazinone, 1,2,4-triazinone, 1,3,5-triazinone and pyridone and wherein RT is as defined in any of the embodiments or aspects defined hereinbefore or hereinafter.

In another embodiment HET-2B is selected from thiopyrimidone, thiopyridazinone, thiopyrazinone, thio-1,2,3-triazinone, thio-1,2,4-triazinone, thio-1,3,5-triazinone and thiopyridone and wherein RT is as defined in any of the embodiments or aspects defined hereinbefore or hereinafter.

In one aspect RT is preferably selected from a substituent from the group (RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or,

(RTa2) (1-4C)alkylamino, di-(1-4C)alkylamino and (2-4C)alkenylamino;

(RTh1) a (1-4C)alkyl group which is optionally substituted by one substituent selected from hydroxy, (1-4C)alkoxy, (1-4C)alkylthio, cyano and azido; or

(RTb2) a (1-4C)alkyl group which is optionally substituted by one substituent selected from (2-4C)alkenyloxy, (3-6C)cycloalkyl and (3-6C)cycloalkenyl;

and wherein at each occurrence of an RT substituent containing an alkyl, alkenyl, alkynyl cycloalkyl or cycloalkenyl moiety in (RTa1) or (RTa2) or (RTh1) or (RTb2) each such moiety is optionally substituted on an available carbon atom with one, two, three or more substituents independently selected from F, CL Br, OH and CN.

In another aspect RT is preferably selected from a substituent from the group:

(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano, and nitro; or

(RTh1) a (1-4C)alkyl group which is optionally substituted by one substituent selected from hydroxy, (1-4C)alkoxy, (1-4C)alkylthio, cyano and azido;

and wherein at each occurrence of an RT substituent containing an alkyl, alkenyl, alkynyl, cycloalkyl or cycloalkenyl moiety in (RTa1) or (RTh1) each such moiety is optionally substituted on an available carbon atom with one, two, three or more substituents independently selected from F, Cl, Br, and CN.

In a further aspect RT is most preferably
(a) hydrogen; or
(b) halogen, in particular fluorine, chlorine, or bromine; or
(c) cyano; or
(d) (1-4C)alkyl, in particular methyl; or
(e) monosubstituted (1-4C)alkyl, in particular fluoromethyl, choromethyl, bromomethyl, cyanomethyl, azidomethyl, hydroxymethyl; or
(f) disubstituted (1-4C)alkyl, for example difluoromethyl, or trisubstituted (1-4C)alkyl, for example trifluoromethyl.

In one embodiment is provided a compound of formula (I) or a pharmaceutically-acceptable salt or an in-vivo hydrolysable ester thereof, wherein group C is represented by group D, R₂b and R₆b are independently H or F; A and B are both oxazolidinones; R₁a and R₁b are independently selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by group B, R₂b and R₆b are independently H or F; A and B are both oxazolidinones; R₁a and R₁b are independently selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by group H, R₂b and R₆b are independently H or F; A and B are both oxazolidinones; R₁a and R₁b are independently selected from OH, —NHCOMe, —NHCOcyclopropyl, —NH(C═S)OMe and —NHCOOMe.

In another embodiment is provided a compound of formula (I) or a pharmaceutically-acceptable salt or an in-vivo hydrolysable ester thereof, wherein group C is represented by group D, R₂b and R₆b are independently H or F; A and B are both oxazolidinones; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl

In another embodiment is provided a compound of formula (I) or a pharmaceutically-acceptable salt or an in-vivo hydrolysable ester thereof, wherein group C is represented by group E, R₂b and R₆b are independently H or F; A and B are both oxazolidinones; R₁a and R₁b are independently selected from —N(—R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group H, R₂b and R₆b are independently H or F; A and B are both oxazolidinones; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by group D, R₂b and R₆b are independently H or F; A is an isoxazoline and B is an oxazolidinone; R₁a and R₁b are independently selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by group E, R₂b and R₆b are independently H or F; A is an isoxazoline and B is an oxazolidinone; R₁a and R₁b are independently selected from OH, —NHCOMe, —NHCOcyclopropyl, —NH(C—S)OMe and —NHCOOMe.

In one embodiment is provided a compound of formula (I) or a pharmaceutically-acceptable salt or an in-vivo hydrolysable ester thereof, wherein group C is represented by group H, R₂b and R₆b are independently H or F; A is an isoxazoline and B is an oxazolidinone; R₁a and R₁b are independently selected from OH, —NHCOMe, —NHCOcyclopropyl, —NH(C═S)OMe and —NHCOOMe.

In another embodiment is provided a compound of formula (I) or a pharmaceutically-acceptable salt or an in-vivo hydrolysable ester thereof, wherein group C is represented by group D, R₂b and R₆b are independently H or F; A is an isoxazoline and B is an oxazolidinone; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group E, R₂b and R₆b are independently H or F; A is an isoxazoline and B is an oxazolidinone; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group H, R₂b and R₆b are independently H or F; A is an isoxazoline and B is an oxazolidinone; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by group D, R₂b and R₆b are independently H or F; B is an isoxazoline and A is an oxazolidinone; R₁a and R₁b are independently selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by group E, R₂b and R₆b are independently H or F; B is an isoxazoline and A is an oxazolidinone; R₁a and R₁b are independently selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by group H, R₂b and R₆b are independently H or F; B is an isoxazoline and A is an oxazolidinone; R₁a and R₁b are independently selected from OH, —NHCOMe, —NHCOcyclopropyl, —NH(C═S)OMe and —NHCOOMe.

In another embodiment is provided a compound of formula (I) or a pharmaceutically-acceptable salt or an in-vivo hydrolysable ester thereof, wherein group C is represented by group D, R₂b and R₆b are independently H or F; B is an isoxazoline and A is an oxazolidinone; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group E, R₂b and R₆b are independently H or F; B is an isoxazoline and A is an oxazolidinone; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group H, R₂b and R₆b are independently H or F; B is an isoxazoline and A is an oxazolidinone; R₁a and R₁b are independently selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group D, R₂b and R₆b are independently H or F; A and B are both oxazolidinones; R₁a is selected from OH, —NHCOMe, —NHCOcyclopropyl, —NH(C═S)OMe and —NHCOOMe and R₁b is selected from —N(R₅)-HET-1A and HET-2A in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group E, R₂b and R₆b are independently H or F; A and B are both oxazolidinones; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group H, R₂b and R₆b are independently H or F; A and B are both oxazolidinones; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group D, R₂b and &b are independently H or F; B is an isoxazoline and A is an oxazolidinone; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group E, R₂b and R₆b are independently H or F; B is an isoxazoline and A is an oxazolidinone; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group H, R₂b and R₆b are independently H or F; B is an isoxazoline and A is an oxazolidinone; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group D, R₂b and R₆b are independently H or F; A is an isoxazoline and B is an oxazolidinone; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group E, R₂b and R₆b are independently H or F; A is an isoxazoline and B is an oxazolidinone; R₁a is selected from OH, —NHCOMe, —NHCOcyclopropyl, —NH(C—S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In another embodiment is provided a compound of formula (I) or a pharmaceutically-acceptable salt or an in-vivo hydrolysable ester thereof, wherein group C is represented by group H, R₂b and R₆b are independently H or F; A is an isoxazoline and B is an oxazolidinone; R₁a is selected from OH, —NHCOMe, —NHCOcyclopropyl, —NH(C═S)OMe and —NHCOOMe and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

When group C is represented by group D, L or M, preferably R₃a is methoxy, methyl or fluoro and R₅a is hydrogen.

When group C is group represented by E, F or G, preferably R₃a is methoxy, methyl or fluoro.

When group C is group represented by H, J, or N, preferably R₃a is methoxy, methyl or fluoro and R₂a′ and R₆a′ are hydrogen; or R₃a and R₂a′ are hydrogen and R₆a′ is methyl or methoxy, particularly methyl.

When group C is group represented by I, K, or 0, preferably R₃a′ is methoxy or methyl and R₅a′ is hydrogen.

In all of the above definitions, aspects and embodiments the preferred compounds are as shown in formula (Ia), i.e. the pharmaceutically active enantiomer.

Particular compounds of the present invention include each individual compound described in the Examples, especially Examples 2 and 4.

Process Section:

In a further aspect the present invention provides a process for preparing a compound of invention or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoro acetic acid, or for example a benzyl group which may be removed, for example, by hydrogenation over a catalyst such as palladium-carbon. Resins may also be used as a protecting group.

The protecting groups may be removed at any convenient stage in the synthesis using conventional techniques well known in the chemical art.

A compound of the invention, or a pharmaceutically-acceptable salt or an in-vivo hydrolysable ester thereof, may be prepared by any process known to be applicable to the preparation of chemically-related compounds. Such processes, when used to prepare a compound of the invention, or a pharmaceutically-acceptable salt or an in-vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March or Houben-Weyl, Methoden der Organischen Chemie). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the certain Patent Application Publications, the contents of the relevant process sections of which are hereby incorporated herein by reference; for example WO 94-13649; WO 98-54161; WO 99-64416; WO 99-64417; WO 00-21960; WO 01-40222.

The skilled organic chemist will be able to use and adapt the information contained and referenced within the above references, and accompanying Examples therein and also the Examples herein, to obtain necessary starting materials, and products. For example, the skilled chemist will be able to apply the teaching herein for compounds of formula (I) in which two central phenyl groups are present (that is when group C is group D) to prepare compounds in which group C is any of groups E to 0 as hereinbefore defined. Similarly, in the processes illustrated below the skilled chemist will be able to apply the teaching as necessary to prepare compounds in which for instance both rings A and B are isoxazoline and those compounds in which one of rings A and B is isoxazoline and the other oxazolidinone.

Thus, the present invention also provides that the compounds of the invention and pharmaceutically-acceptable salts and in-vivo hydrolysable esters thereof, can be prepared by a process (a) to (h); and thereafter if necessary:

i) removing any protecting groups;

ii) forming a pro-drug (for example an in-vivo hydrolysable ester); and/or

iii) forming a pharmaceutically-acceptable salt;

wherein said processes (a) to (h) are as follows (wherein the variables are as defined above unless otherwise stated):

a) by modifying a substituent in, or introducing a substituent into another compound of the invention by using standard chemistry (see for example, Comprehensive Organic Functional Group Transformations (Pergamon), Katritzky, Meth-Cohn & Rees or Advanced Organic Chemistry (Wiley-Interscience), Jerry March or Houben-Weyl, Methoden der Organischen Chemie)); for example:

an acylamino group may be converted into a thioacylamino group;

an acylamino group or thioacylamino group may be converted into another acylamino or thioacylamino; heterocyclyl for instance tetrazolyl or thiazolyl, or heterocyclylamino group (optionally substituted or protected on the amino-nitrogen atom), a heterocyclyl group linked through nitrogen (optionally substituted on a carbon other than a carbon adjacent to the linking nitrogen atom), for instance an optionally 4-substituted 1,2,3-triazol-1-yl group; or an amidino group; such conversions of the acylamino group taking place either directly or through through the intermediacy of one or more derivatives such as an amino group;

an acyloxy group may be converted into a hydroxy group or into the groups that may be obtained from a hydroxy group (either directly or through the intermediacy of a hydroxy group);

an alkyl halide such as alkylbromide or alkyliodide may be converted into an alkyl fluoride or nitrile;

an alkyl sulfonate such as alkyl methanesulfonate may be converted into an alkyl fluoride or nitrile;

an alkylthio group such as methylthio may be converted into a methanesulfinyl or methanesulfonyl group,

an arylthio group such as phentlthio may be converted into a benzenesulfinyl or benzenesulfonyl group,

an amidino or guanidino group may be converted into a range of 2-substituted 1,3-diazoles and 1,3-diazines

an amino group may be converted for instance into acylamino or thioacylamino for instance an acetamide (optionally substituted), alkyl- or dialkyl-amino and thence into a further range of N-alkyl-amine derivatives, sulfonylamino, sulfinylamino, amidino, guanidino, arylamino, heteroarylamino, N-linked heterocyclic for instance an optionally 4-substituted 1,2,3-triazol-1-yl group;

an aryl- or heteroary-halide group such as an aryl- or hetero-aryl chloride or bromide or iodide may be converted by transition metal mediated coupling, especially Pd(0) mediated coupling into a range of aryl-, heteroaryl, alkenyl, alkynyl acyl, alkylthio, or alkyl- or dialkyl-amino substituted aryl or heteroaryl groups;

an aryl- or heteroary-sulfonate group such as an aryl- or hetero-aryl trifluoromethanesulfonate may be converted by transition metal mediated coupling, especially Pd(0) mediated coupling into a range of aryl-, heteroaryl alkenyl, alkynyl, acyl, alkylthio, or alkyl- or dialkyl-amino substituted aryl or heteroaryl groups;

an aryl- or heteroary-halide group such as an aryl- or hetero-aryl chloride or bromide or iodide may be converted by transition metal mediated coupling, especially Pd(0) mediated coupling into a range of trialkyltin, dialkylboronate, trialkoxysilyl, substituted aryl or heteroaryl groups useful as intermediates for the synthesis of compounds of the invention;

an azido group may be converted for instance into a 1,2,3-triazolyl or amine and thence by methods that are well known in the art into any of the range common amine derivatives such as acylamino for instance acetamido group;

a carboxylic acid group may be converted into trifloromethyl, hydroxymethyl, alkoxycarbonyl aminocarbonyl optionally substituted on nitrogen, formyl, or acyl groups;

a cyano group may be converted into a tetrazole, or an imidate, an amidine, an amidrazone, an N-hydroxyamidrazone, an amide, a thioamide, an ester, or an acid and thence by methods that are well known in the art into any of the range of heterocycles derived from such nitrile derivatives;

a hydroxy group may be converted for instance into an alkoxy, cyano, azido, alkylthio, keto and oximino, fluoro, bromo, chloro, iodo, alkyl- or aryl-sulfonyloxy for instance trifluoromethanesulfonate, methanesulfonate, or tosylsulfonate, silyloxy; acylamino or thioacylamino, for instance an acetamide (optionally substituted or protected on the amido-nitrogen atom); acyloxy, for instance an acetoxy; phosphono-oxy, heterocyclylamino (optionally substituted or protected on the amino-nitrogen atom), for instance an isoxazol-3-ylamino or a 1,2,5-thiadiazol-3-ylamino; heterocyclyl linked through nitrogen (optionally substituted on a carbon other than a carbon atom adjacent to the linking nitrogen ring atom), for instance an optionally 4-substituted 1,2,3-triazol-1-yl; or amidino, for instance an 1-(N-cyanoimino)ethylamino group; such conversions of the hydroxy group taking place directly (for instance by acylation or Mitsunobu reaction) or through the intermediacy of one or more derivatives (for instance a mesylate or an azide);

a keto group may be converted into a hydroxy, thiocarbonyl, oximino, or difluoro group; a nitro-group may be converted into an amino group and thence by methods that are well known in the art into any of the range common amine derivatives such as acylamino for instance acetamido group;

a silyloxy group may be converted into a hydroxy group or into the groups that may be obtained from a hydroxy group (either directly or through the intermediacy of a hydroxy group);

an optionally substituted aromatic or heteroaromatic ring C′may be converted into another aromatic or heteroaromatic ring C′ by introduction of a new substituent (R₂a to R₆a or R₂a′ or R₆a′) or by refunctionalisation of an existing substituent (R₂a to R₆a or R₂a′ or R₆a′);

a heterocyclylamino group (optionally substituted or protected on the amino-nitrogen atom) may be converted into another heterocyclyl amino group (optionally substituted or protected on the amino-nitrogen atom) by refunctionalisation, for instance by protection or deprotection, of the amino-nitrogen atom, by introduction of a new ring substituent, or by refunctionalisation of an existing ring substituent; or

a heterocyclyl group linked through nitrogen (optionally substituted on a carbon other than a carbon atom adjacent to the linking nitrogen ring atom) may be converted into another heterocyclyl group linked through nitrogen (optionally substituted on a carbon other than a carbon atom adjacent to the linking nitrogen ring atom) by introduction of a new ring substituent or by refunctionalisation of an existing ring substituent, for instance by modifying the 4-substituent of a 4-substituted 1,2,3-triazol-1-yl group.
For instance, examples drawn from the methods for conversion of a hydroxy group into an optionally substituted triazole group are illustrated by the scheme:

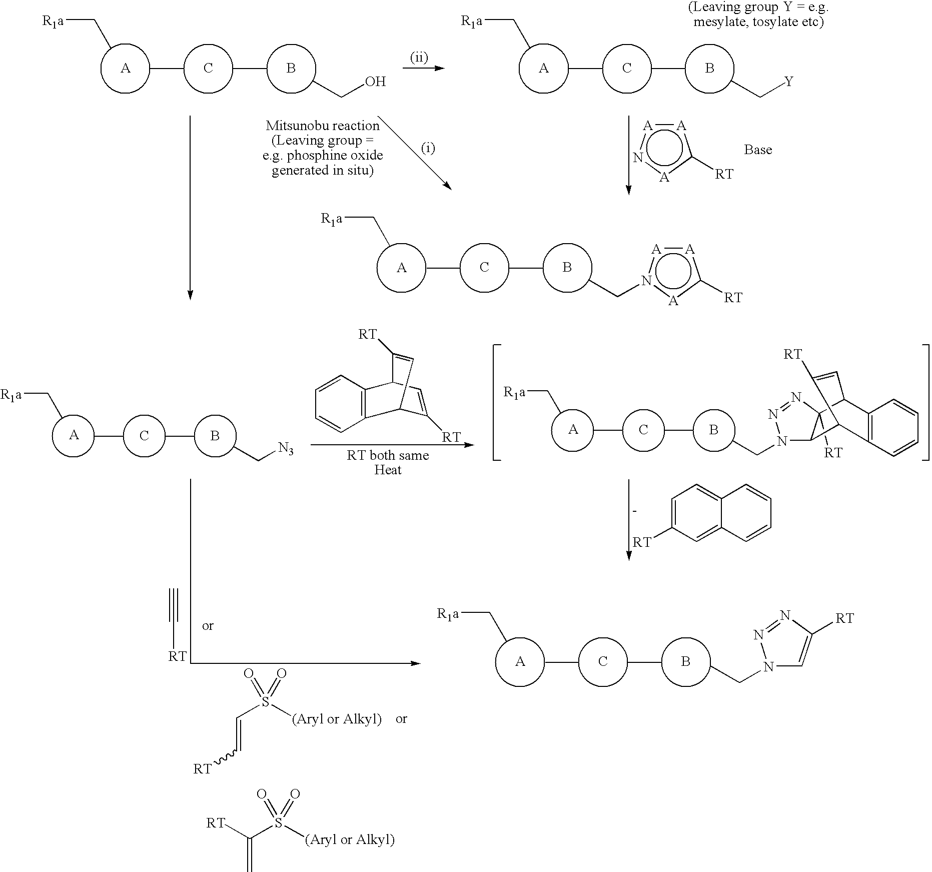

Examples drawn from the range of regioselective methods that proceed under very mild conditions are further illustrated by processes (f), (g), and (h).
b) by reaction of a molecule of a compound of formula (IIa) (wherein X is a leaving group useful in palladium coupling, for example boronate, trimethyl tin, iodo and bromo) with a molecules of a compound of formula (IIb) (wherein X′ is a leaving group useful in palladium coupling, for example boronate, trimethyl tin, iodo and bromo) wherein X and X′ are chosen such that an aryl-aryl, heteroaryl-aryl, or heteroaryl-heteroaryl bond replaces the aryl-X (or heteroaryl-X) and aryl-X′ (or heteroaryl-X′) bonds. Such methods are now well known, see for instance S. P. Stanforth, Catalytic Cross-Coupling Reactions in Biaryl Synthesis, Tetrahedron, 541998, 263-303. 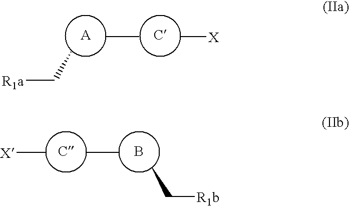
The leaving groups X and X′ may be chosen to be the same and lead to symmetrical molecules of formula (I) or different and chosen to lead to symmetrical or unsymmetrical molecules of formula (I).
For example 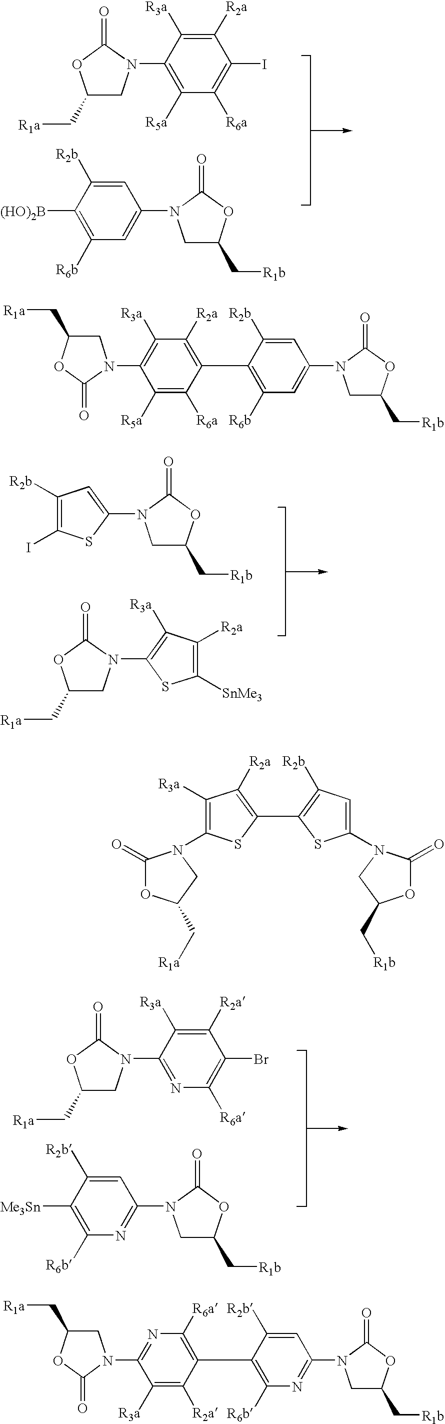 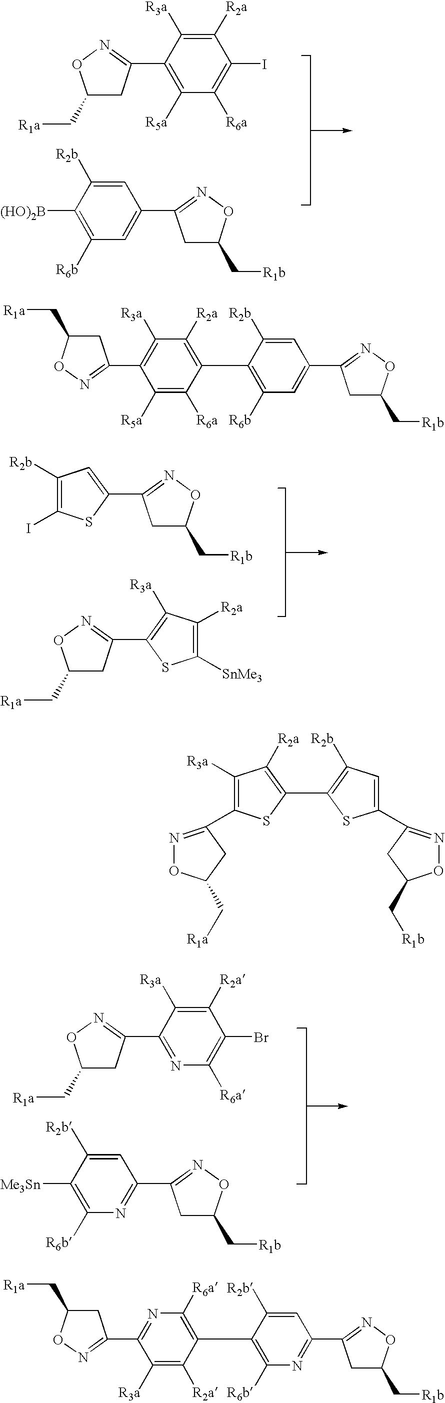
Similarly, this chemistry may be applied to two dissimilar molecules of formula (II), for example those in which ring A is not the same as ring B, wherein X is suitably selected to enable unsymmetrical coupling so that an aryl-aryl, heteroaryl-aryl, or heteroaryl-heteroaryl bond replaces the aryl-X (or heteroaryl-X) and the aryl-X′ (or heteroaryl-X′) bonds.
For example 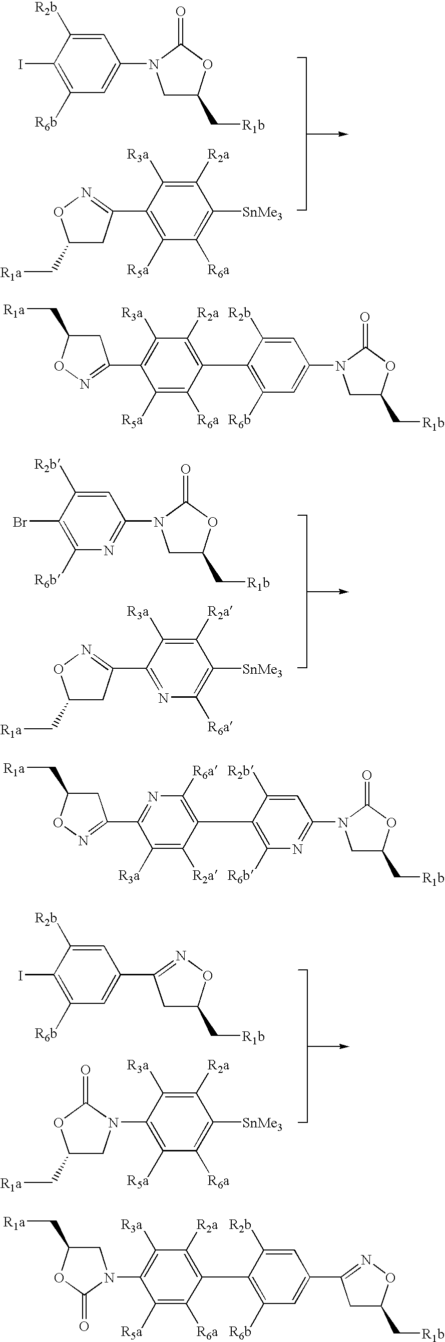 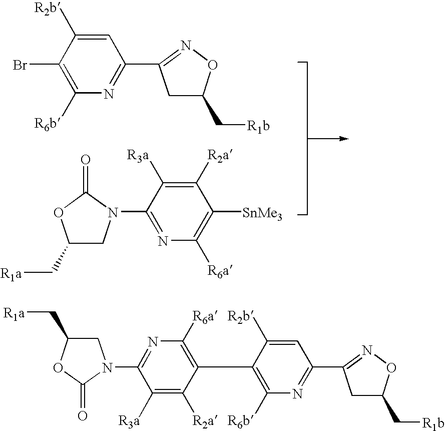
Furthermore, this chemistry may also be applied to two dissimilar molecules of formula (II), for example those in which ring C′ is not the same as ring C″, wherein X and X′ are suitably selected to enable unsymmetrical coupling so that an aryl-aryl, heteroaryl-aryl, or heteroaryl-heteroaryl bond replaces the two different aryl-X (or heteroaryl-X) and the aryl-X′ (or heteroaryl-X′) bonds.
For example 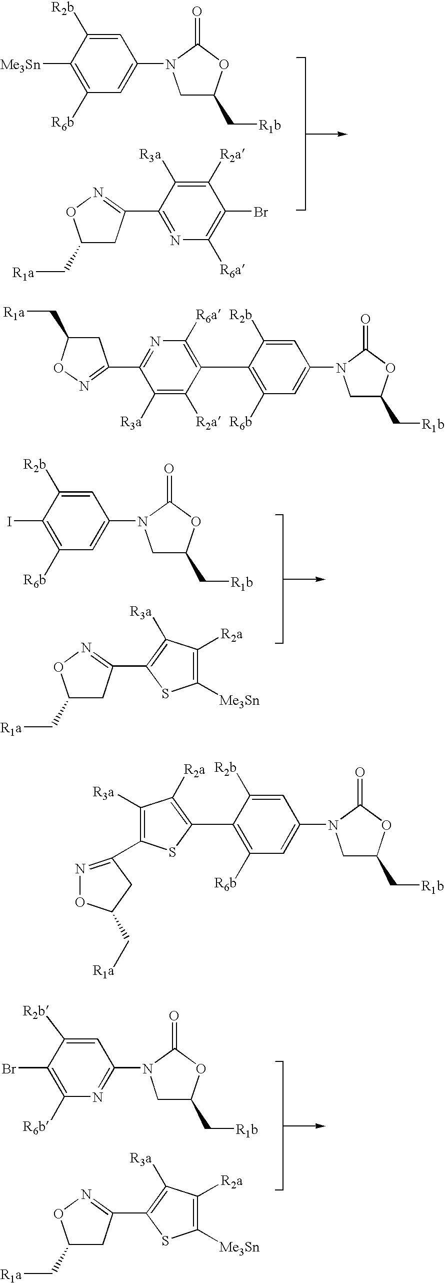 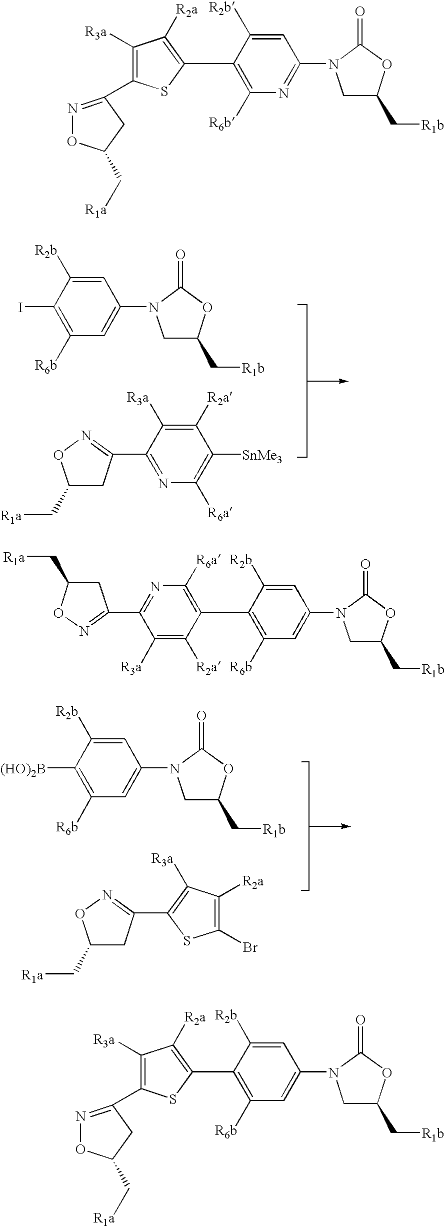 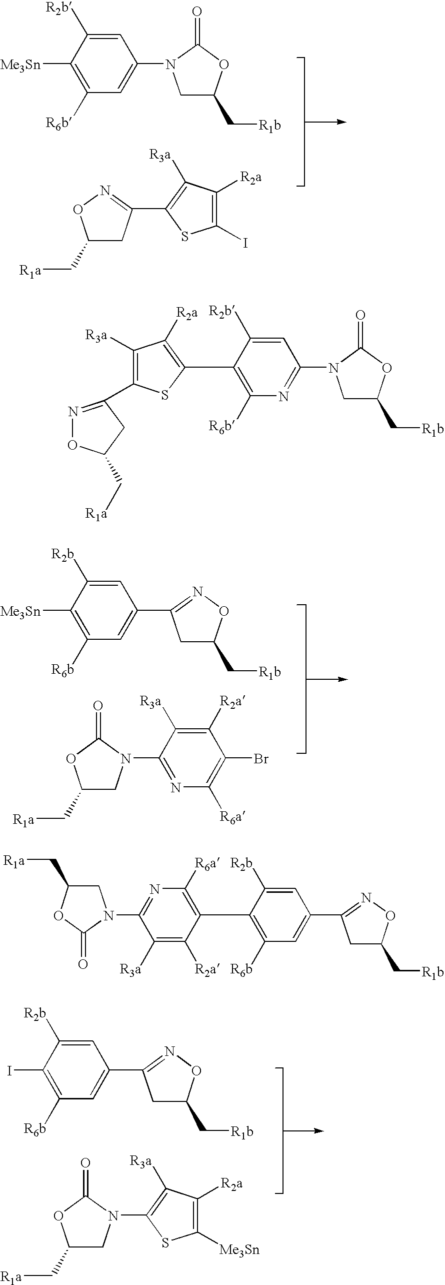 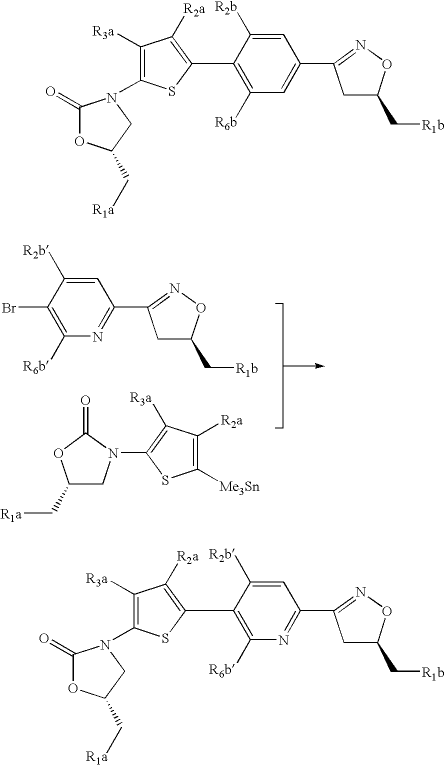
The aryl isoxazolines and aryl oxazolidiones required as reagents for process b) or as intermediates for the preparation of reagents for process b) may be prepared by standard organic methods, for instance by methods analogous to those set out in process sections c) to h). Methods for the introduction and interconversion of Groups X and X′ are well known in the art.
c) by reaction of a (hetero)biaryl derivative (IIIa) or (IIIb) carbamate with an appropriately substituted oxirane to form an oxazolidinone ring at the undeveloped aryl position. 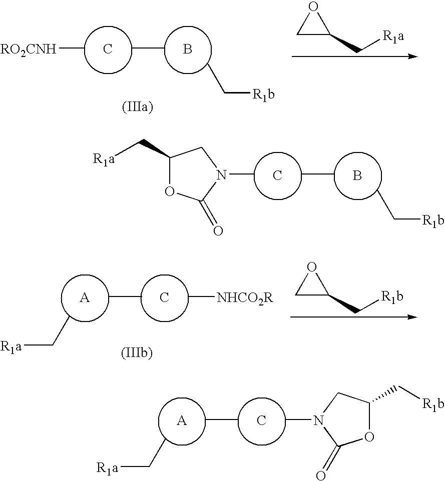
Variations on this process in which the carbamate is replaced by an isocyanate or by an amine or/and in which the oxirane is replaced by an equivalent reagent X—CH₂CH(O-optionally protected)CH₂R₁a or X—CH₂CH(O-optionally protected)CH₂R₁b where X is a displaceable group are also well known in the art.
For example, 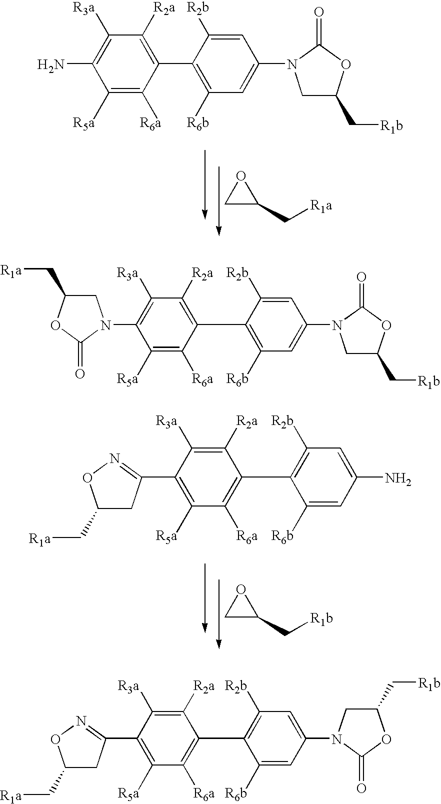
d) by reaction of a (hetero)biaryl derivative (IVa) or (IVb) to form an isoxazoline ring at the undeveloped aryl position. 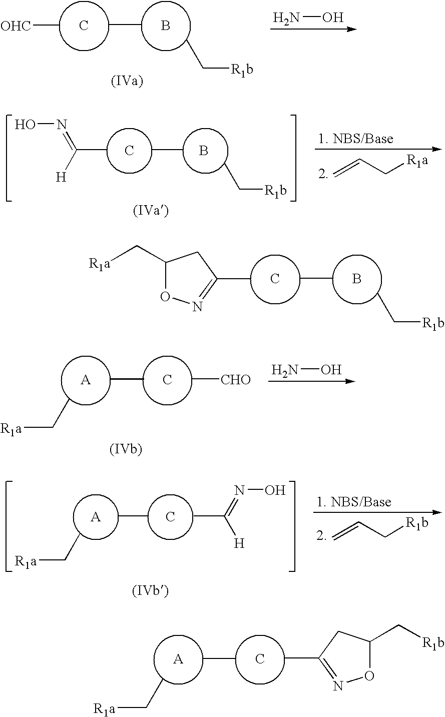
Variations on this process in which the reactive intermediate (a nitrile oxide IVa″ or IVb″) is obtained other than by oxidation of an oxime (IVa′) or (IVb′) are well known in the art. 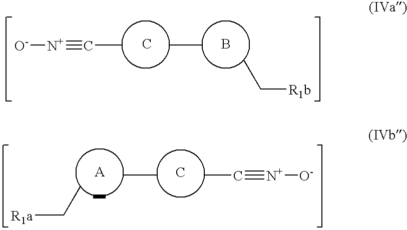
For example, oxidation of an appropriately substituted biphenylcarboxaldehyde oxime in the presence of an appropriately substituted allyl derivative gives an isoxazoline of the required structure. 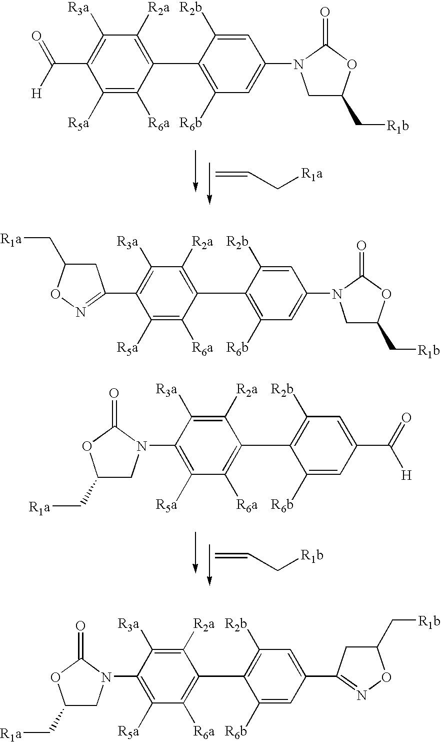
Enantioselective synthesis of 2-isoxazolines via asymmetric cycloaddition of nitrile oxides to olefins has been achieved by the use of chiral auxiliaries. For instance, when the alcohol is an allyl alcohol the desired stereochemistry at ring B can be obtained in reactions conducted in the presence of (R,R)-diisopropyl tartrate (or (S,S)-diisopropyl tartrate depending on the desired stereochemistry) as a chiral auxiliary (Yutaka Ukaji et al. Chem. Letters, 1993, 1847-1850). Other chiral auxiliaries may also be employed with other olefins (see for instance Takahiko Akayama et al, Tet. Letters, 1992, 33, 5763-5766; and Jeffrey Stack et al., Tetrahedron, 1993, 49, 995-1008 and references therein). 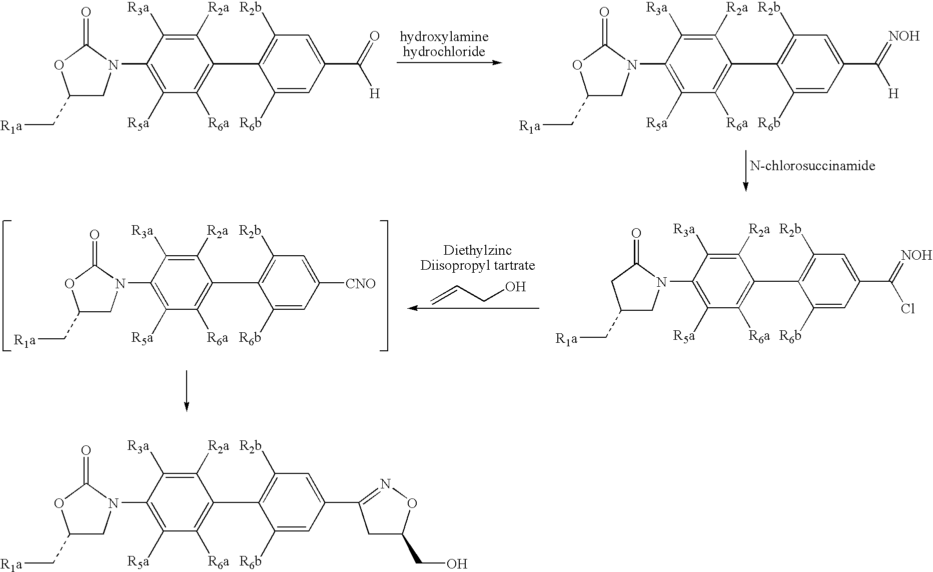
(e) for HET as optionally substituted 1,2,3-triazoles, compounds of the formula (I) may be made by cycloaddition via the azide (wherein e.g. Y in (II) is azide) to acetylenes, or to acetylene equivalents such as optionally substituted cylcohexa-1,4-dienes or optionally substituted ethylenes bearing eliminatable substituents such as arylsulfonyl; or
(f) for HET as 4-substituted 1,2,3-triazole compounds of formula (I) may be made by reacting aminomethyloxazolidinones with 1,1-dihaloketone sulfonylhydrazones (Sakai, Kunihazu; Hida, Nobuko; Kondo, Kiyosi; Bull Chem. Soc. Jpn., 59, 1986, 179-183; Sakai, Kunikazu; Tsunemoto, Daiei; Kobori, Takeo; Kondo, Kiyoshi; Hido, Noboko EP 103840 A2 19840328); for instance 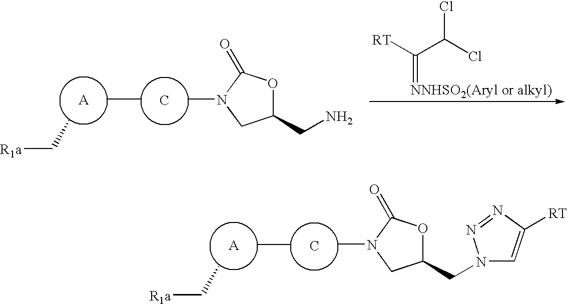
(g) for HET as 4-substituted 1,2,3-triazole compounds of formula (I) may also be made by reacting azidomethyl oxazolidinones with terminal alkynes using Cu(I) catalysis in e.g. aqueous alcoholic solution at ambient temperatures to give 4-substituted 1,2,3-triazoles (V. V. Rostovtsev, L. G. Green, V. V. Fokin, and K. B. Sharpless, Angew. Chem Int. Ed., 2002, 41, 2596-2599): for instance 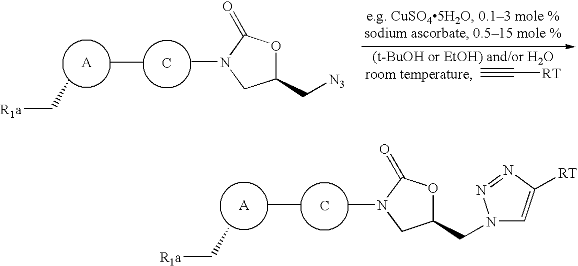
(h) for HET as 4-halogenated 1,2,3-triazole compounds of formula (I) may also be made by reacting azidomethyl oxazolidinones with halovinylsulfonyl chlorides at a temperature between 0° C. and 100° C. either neat or in an inert diluent such as chlorobenzene, chloroform or dioxan; for instance. 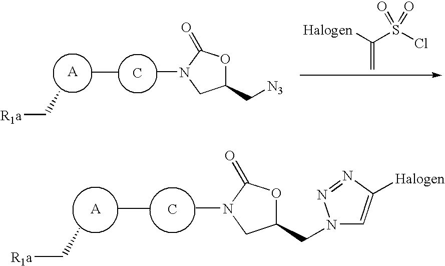
and thereafter if necessary:
i) removing any protecting groups;
ii) forming a pro-drug (for example an in-vivo hydrolysable ester); and/or
iii) forming a pharmaceutically-acceptable salt.

The removal of any protecting groups, the formation of a pharmaceutically-acceptable salt and/or the formation of an in-vivo hydrolysable ester are within the skill of an ordinary organic chemist using standard techniques. Furthermore, details on the these steps, for example the preparation of in-vivo hydrolysable ester prodrugs has been provided, for example, in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention there is provided a compound of the invention, or a pharmaceutically-acceptable salt, or in-vivo hydrolysable ester thereof for use in a method of treatment of the human or animal body by therapy.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt, or in-vivo hydrolysable ester thereof.

The invention also provides a compound of the invention, or a pharmaceutically-acceptable salt, or in-vivo hydrolysable ester thereof, for use as a medicament; and the use of a compound of the invention of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the invention, an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the invention, an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition of this invention may also contain (ie through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents. Compounds of this invention may also be co-formulated or co-administered with a vitamin, for example Vitamin B, such as Vitamin B2, Vitamin B6, Vitamin B12 and folic acid. Compounds of the invention may also be formulated or co-administered with cyclooxygenase (COX) inhibitors, particularly COX-2 inhibitors.

In one aspect of the invention, a compound of the invention is co-formulated with an antibacterial agent which is active against gram-positive bacteria.

In another aspect of the invention, a compound of the invention is co-formulated with an antibacterial agent which is active against gram-negative bacteria.

In another aspect of the invention, a compound of the invention is co-administered with an antibacterial agent which is active against gram-positive bacteria.

In another aspect of the invention, a compound of the invention is co-administered with an antibacterial agent which is active against gram-negative bacteria.

The compositions of the invention may be obtained by conventional procedures using conventional pharmaceutical excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 50 mg to 5 g of active agent compounded with an appropriate and convenient amount of excipients which may vary from about 5 to about 98 percent by weight of the total composition. Dosage unit forms will generally contain about 200 mg to about 2 g of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mgkg⁻¹ to 20 mgkg⁻¹ of a compound of this invention, the composition being administered 1 to 4 times per day. In another embodiment a daily dose of 5 mgkg⁻¹ to 20 mgkg⁻¹ of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

In the above other, pharmaceutical composition, process, method, use and medicament manufacture features, the alternative and preferred embodiments of the compounds of the invention described herein also apply.

Antibacterial Activity:

The pharmaceutically-acceptable compounds of the present invention are useful antibacterial agents having a good spectrum of activity in vitro against standard Gram-positive organisms, which are used to screen for activity against pathogenic bacteria Notably, the pharmaceutically-acceptable compounds of the present invention show activity against enterococci, pneumococci and methicillin resistant strains of S. aureus and coagulase negative staphylococci together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CPU/well.

For example, the following results were obtained for the compound of Example 4:

| Organism | | MIC (μg/ml) |
|---|---|---|
| Staphylococcus aureus: | MSQS | 0.5 |
| | MRQR | 0.5 |
| Streptococcus pneumoniae | | 0.13 |
| Haemophilus influenzae | | 4 |
| Moraxella catarrhalis | | 0.5 |
| Enterococcus faecium | | 0.5 |
| Linezolid Resistant Streptococcus pneumoniae | | 1 |

MSQS = methicillin sensitive and quinolone sensitive
MRQR = methicillin resistant and quinolone resistant

Certain intermediates and/or Reference Examples described hereinafter are within the scope of the invention and may also possess useful activity, and are provided as a further feature of the invention.

The invention is now illustrated but not limited by the following Examples in which unless otherwise stated:—

(i) evaporations were carried out by rotary evaporation in-vacuo and work-up procedures were carried out after removal of residual solids by filtration;

(ii) operations were carried out at ambient temperature, that is typically in the range 18-26° C. and without exclusion of air unless otherwise stated, or unless the skilled person would otherwise work under an inert atmosphere;

(iii) column chromatography (by the flash procedure) was used to purify compounds and was performed on Merck Kieselgel silica (Art. 9385) unless otherwise stated;

(iv) yields are given for illustration only and are not necessarily the maximum attainable;

(v) the structure of the end-products of the invention were generally confirmed by NMR and mass spectral techniques [proton magnetic resonance spectra were generally determined in DMSO-d₆ unless otherwise stated using a Varian Gemini 2000 spectrometer operating at a field strength of 300 MHz, or a Bruker AM250 spectrometer operating at a field strength of 250 MHz; chemical shifts are reported in parts per million downfield from tetramethysilane as an internal standard (δ scale) and peak multiplicities are shown thus: s, singlet; d, doublet; AB or dd, doublet of doublets; dt, doublet of triplets; di, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected]; optical rotations were determined at 589 nm at 20° C. for 0.1M solutions in methanol using a Perkin Elmer Polarimeter 341;

(vi) each intermediate was purified to the standard required for the subsequent stage and was characterised in sufficient detail to confirm that the assigned structure was correct; purity was assessed by HPLC, TLC, or NMR and identity was determined by intra-red spectroscopy (IR), mass spectroscopy or NMR spectroscopy as appropriate;

(vii) in which the following abbreviations may be used:—

DMP is N,N-dimethylformamide; DMA is N,N-dimethylacetamide; TLC is thin layer chromatography; BPLC is high pressure liquid chromatography; MPLC is medium pressure liquid chromatography; DMSO is dimethylsulfoxide; CDCl₃ is deuterated chloroform; MS is mass spectroscopy; ESP is electrospray; EI is electron impact; CI is chemical ionisation; APCI is atmospheric pressure chemical ionisation; EtOAc is ethyl acetate; MeOH is methanol; phosphoryl is (HO)₂—P(O)—O—; phosphiryl is (HO)₂—P—O—; Bleach is “Clorox” 6.15% sodium hypo chlorite; THF is tetrahydrofuran; TFA is trifluoro acetic acid; EDAC is

(viii) temperatures are quoted as ° C.

### EXAMPLE 1

### (5R)-3-{4′-[5-({[tert-Butyl(dimethyl)silyl]oxy}methyl)-4,5-dihydroisoxazol-3-yl]-2-fluoro-3′-methoxy-1,1′-biphenyl-4-yl}-5-(1H-1,2,3-triazol-1-ylmethyl)-1,3-oxazolidin-2-one

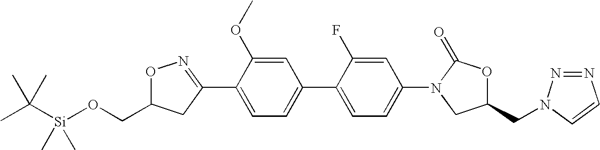 A stirred mixture of (5R)-3-(3-Fluoro-4-iodophenyl)-5-(1H-1,2,3-triazol-1-ylmethyl)-1,3-oxazolidin-2-one (186 mg, 0.48 mmol), 5-({[tert-butyl(dimethyl)silyl]oxy}methyl)-3-[2-methoxy-4-(trimethylstannyl)phenyl]-4,5-dihydroisoxazole (255 mg, 0.53 mmol) and copper (I) iodide (38 mg, 0.20 mmol) in dry 1-methyl-2-pyrrolidinone (2 mL) was degassed and then treated under argon with tetrakis(triphenylphosphine)palladium(0) (55 mg, 0.05 mmol). The reaction mixture stirred for 16 hours at 90° C. and then cooled and partitioned between water (20 mL) and ethyl acetate (20 mL). The ethyl acetate layer was separated, dried over magnesium sulphate, filtered and the product was concentrated in vacuo onto Isolute HM-N (4 mL). The product was purified by by chromatography on silica-gel [SiO₂ 20 g bond elut: elution gradient from 10% to 50% iso-propanol:hexanes] to give the title compound (89 mg, 32%).

MS (ESP+): (M+H)⁺582.11 for C₂₉H₃₆FN₅O₅Si

NMR (DMSO-d₆) δ: 0.07 (s, 3H); 0.09 (s, 3H); 0.86 (s, 9H); 3.24 (dd, 1H); 3.47 (dd, 1H); 3.69 to 3.79 (m, 2H); 3.80 (s, 3H); 3.96 (dd, 1H); 4.31 (t, 1H); 4.85 (m, 1H); 4.87 (d, 2H); 5.19 (m, 1H); 7.30 to 7.42 (m, 5H); 7.49 (m, 1H); 7.80 (m, 1H); 8.21 (s, 1H).

The intermediates for these compounds were prepared as follows:—

### 4-Bromo-2-methoxybenzaldehyde

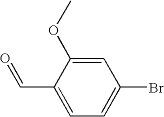 A stirred solution of 4-bromo-2-hydroxybenzaldehyde (1.035 g, 5.12 mmol) in anhydrous acetone (75 mL) was treated with potassium carbonate (0.865 g, 6.26 mmol) and dimethylsulphate (0.44 mL, 4.6 mmol) and then heated under reflux for 90 minutes. The reaction mixture was filtered and the product was then concentrated in vacuo onto Isolute HM-N (10 mL). The product was purified by by chromatography on silica-gel [SiO₂ 50 g bond elut: elution gradient 0% to 25% ethyl acetate:hexanes] to give the title compound (0.616 g, 56%).

MS (APCI+): (M+acetonitrile)⁺256 & 258 for C₈H₇BrO₂

NMR (DMSO-d₆) δ: 3.95 (s, 3H); 7.18 (dd, 1H); 7.28 (d, 1H); 7.98 (d, 1H); 9.94 (s, 1H).

### 4-Bromo-2-methoxybenzaldehyde oxime

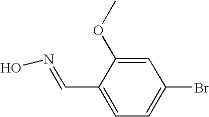

A stirred solution of 4-bromo-3-methoxybenzaldehyde (0.616 g, 2.86 mmol) in methanol (20 mL) and water (2 mL) was treated with hydroxylamine hydrochloride (0.234 g, 3.44 mmol) and sodium carbonate (0.182 g, 1.72 mmol). The reaction mixture was stirred at room temperature for 16 hours then the methanol removed in vacuo. The involatile residue was partitioned between water (100 mL) and ethyl acetate (100 mL). The ethyl acetate layer was separated, dried over magnesium sulphate, filtered, and the product was concentrated in vacuo onto Isolute HM-N (5 mL). The product was purified by by chromatography on silica-gel [SiO₂ 20 g bond elut: elution gradient 0% to 25% ethyl acetate:hexanes] to give the title compound (324 mg 49%).

NMR (DMSO-d₆) δ: 3.90 (s, 3H); 6.83 (dd, 1H); 7.09 (d, 1H); 7.27 (br s, 1H); 7.76 (d, 1H); 8.07 (s, 1H).

### [3-(4-Bromo-2-methoxyphenyl)-4,5-dihydroisoxazol-5-yl]methanol

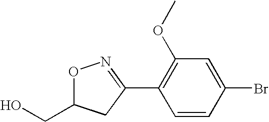 A stirred solution of 4-bromo-2-methoxybenzaldehyde oxime (320 mg, 1.4 mmol) in tetrahydrofuran (2 mL) was treated at room temperature with allyl alcohol (0.14 mL, 2.1 mmol) and then with household bleach (“Clorox” 6.15% sodium hypochlorite; 10 mL). The reaction mixture was stirred at room temperature for 16 hours and then extracted with ethyl acetate (20 mL). The ethyl acetate layer was separated, dried over magnesium sulphate, filtered, and then the product was concentrated in vacuo onto Isolute HM-N (5 mL). The product was purified by column chromatography [SiO₂ 10 g bond elut: elution gradient 50% to 100% ethyl acetate:hexanes] to give the title compound (239 mg 60%). NMR (DMSO-d₆) δ: 3.23 (dd, 1H); 3.39 to 3.55 (m, 3H); 3.89 (s, 3H); 4.74 (m, 1); 5.02 (t, 1H); 7.04 (dd, 1H); 7.23 (d, 1H); 7.86 (d, 1H).

### 3-(4-Bromo-2-methoxyphenyl)-5-({[tert-butyl(dimethyl)silyl]oxy}methyl)-4,5-dihydroisoxazole

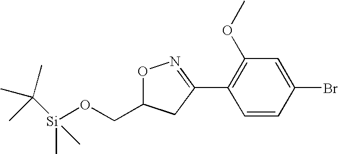 A solution of [3-(4-Bromo-2-methoxyphenyl)-4,5-dihydroisoxazol-5-yl]methanol (0.239 g, 0.84 mmol) in a mixture of triethylamine (0.14 mL, 1.0 mmol) and dichloromethane (10 mL) was treated dropwise during 5 minutes with a solution of tert-butyldimethylsilylchloride (1M, 0.92 mL) in dichloromethane and then with 4-dimethylaminopyridine (10 mg, 0.084 mmol). The reaction mixture was stirred overnight at room temperature and then concentrated in vacuo onto Isolute HM-N (3 mL). The product was purified by chromatography [SiO₂ 20 g bond elut; elution gradient 0% to 25% ethyl acetate:hexanes] to give the title compound (0.27 g 81%) as a white crystalline solid.

NMR (DMSO-d₆) δ: 0.05 (s, 3H); 0.07 (s, 3H); 0.84 (s, 9H); 3.20 (dd, 1H); 3.46 (dd, 1H); 3.67 to 3.79 (m, 2H); 3.88 (s, 3H); 4.79 (m, 1H); 7.02 (dd, 1H); 7.22 (d, 2H); 7.85 (d, 1H).

### 5-({[tert-Butyl(dimethyl)silyl]oxy}methyl)-3-[2-methoxy-4-(trimethylstannyl)phenyl]-4,5-dihydroisoxazole

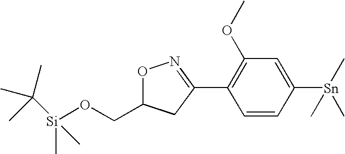 A stirred solution of 3-(4-bromo-2-methoxyphenyl)-5-({[tert-butyl(dimethyl)silyl]oxy}methyl)-4,5-dihydroisoxazole (0.27 g, 0.67 mmol) in dry 1,4-dioxane (6 mL) was degassed and maintained under an atmosphere of argon. The mixture was treated with hexamethylditin (0.265 g, 0.81 mmol) and then with bis(triphenylphosphine)palladium(II) chloride (0.024 g, 0.03 mmol). The reaction mixture was stirred at 90° C. for 180 minutes under an atmosphere of argon. The solvent was removed in vacuo, the crude product was re-dissolved in hexanes (10 mL) and filtered to remove insoluble material. The hexane solution of the product was purified by chromatography [SiO₂ 10 g bond elut: elution gradient 0% to 20% ethyl acetate:hexanes] to give the title compound (0.255 g 78%) as an oil.

MS (ESP+): (M+H)⁺481.89, 483.96, 485.83, 487.83 & 489.90 for C₂₀H₃₅NO₃SiSn

NMR (DMSO-d₆) δ: 0.06 (s, 3H); 0.07 (s, 3H); 0.26 (t, 9H); 0.85 (s, 9H); 3.18 (dd, 1H); 3.44 (dd, 1H); 3.66 to 3.79 (d, 2H); 3.80 (s, 3H); 4.77 (m, 1H); 7.14 to 7.24 (m, 2H); 7.39 (d, 1H).

### Acetic acid (5R)-3-(3-fluorophenyl)-1,3-oxazolidin-2-on-5-ylmethyl ester

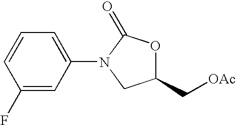 (5R)-3-(3-Fluorophenyl)-5-hydroxymethyl-1,3-oxazolidin-2-one (40 g, 0.189 M, see Upjohn WO 94-13649) was suspended by stirring in dry dichloromethane (400 mL) under nitrogen. Triethylamine (21 g, 0.208 M) and 4-dimethylaminopyridine (0.6 g, 4.9 mM) were added, followed by dropwise addition of acetic anhydride (20.3 g, 0.199 M) over 30 minutes, and stirring continued at ambient temperature for 18 hours. Saturated aqueous sodium bicarbonate (250 mL) was added, the organic phase separated, washed with 2% sodium dihydrogen phosphate, dried (magnesium sulfate), filtered and evaporated to give the desired product (49.6 g) as an oil

MS (ESP): 254 (MH⁺) for C₁₂H₁₂FNO₄

NMR (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).

### Acetic acid (5R)-3-(3-fluoro-4-iodo-phenyl)-1,3-oxazolidin-2-one-5-ylmethyl ester

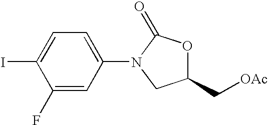 Acetic acid (5R)-3-(3-fluoro-phenyl)-1,3-oxazolidin-2-one-5-ylmethyl ester (15.2 g, 60 mM) was dissolved in a mixture of chloroform (100 mL) and acetonitrile (100 mL) under nitrogen, and silver trifluoroacetate (16.96 g, 77 mM) added. Iodine (18.07 g, 71 nm was added in portions over 30 minutes to the vigorously stirred solution, and stirring continued at ambient temperature for 18 hours. As reaction was not complete, a further portion of silver trifluoroacetate (2.64 g, 12 mM) was added and stirring continued for 18 hours. After filtration, the mixture was added to sodium thiosulfate solution (3%, 200 mL) and dichloromethane (200 ml), and the organic phase separated, washed with sodium thiosulfate (200 mL), saturated aqueous sodium bicarbonate (200 mL), brine (200 mL), dried (magnesium sulfate), filtered and evaporated. The crude product was suspended in isohexane (100 mL), and sufficient diethyl ether added to dissolve out the brown impurity while stirring for 1 hour. The product was isolated by filtration to give the title compound (24.3 g) as a cream solid.

MS (ESP): 380 (MH⁺) for C₁₂H₁₁FINO₄

NMR (MSO-d₆) δ: 2.03 (s, 3H); 3.82 (dd, 1H); 4.15 (t, 1); 4.24 (dd, 1H); 4.30 (dd, 1); 4.94 (m, 1); 7.19 (dd, 1H); 7.55 (dd, 1H); 7.84 (t, 1H).

### (5R)-3-(3-Fluoro-4-iodophenyl)-5-hydroxymethyl-1,3-oxazolidin-2-one

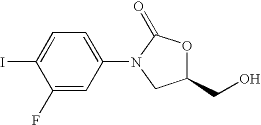 A solution of acetic acid (5R)-3-(3-fluoro-4-iodophenyl)-1,3-oxazolidin-2-one-5-ylmethyl ester (30 g, 79 mM) in a mixture of methanol (800 mL) and dichloromethane (240 mL) was treated at ambient temperature with potassium carbonate (16.4 g, 0.119 mM) for 25 minutes, then immediately neutralised by the addition of acetic acid (10 mL) and water (500 mL). The precipitated product was filtered, washed with water, and then dissolved in dichloromethane (1.2 L) to give a the solution that was washed with saturated sodium bicarbonate and then dried (magnesium sulfate). The solution of product was filtered and evaporated to dryness to give the title compound (23 g).

MS (ESP): 338 (MH⁺) for C₁₀H₉FINO₃

NMR (DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).

### (5R)-5-Azidomethyl-3-(3-fluoro-4-iodophenyl)-1,3-oxazolidin-2-one

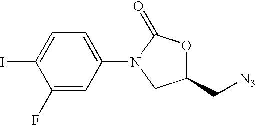 A stirred solution of (5R)-3-(3-fluoro-4-iodophenyl)-5-hydroxymethyl-1,3-oxazolidin-2-one (55.8 g) and triethylamine (46.1 mL) in dry dichloromethane (800 mL) under an atmosphere of dry nitrogen was maintained below room temperature by an ice-bath and treated dropwise with methanesulfonyl chloride (17.9 mL). The stirred reaction mixture was allowed to warm to room temperature during 3 hours and then washed sequentially with water and brine and then dried (Na₂SO₄). Solvent was removed under reduced pressure to give the intermediate mesylate as a yellow solid (68 g) that was used without further purification.
A stirred solution in DMF (800 mL) of a mixture of the intermediate mesylate (68 g) and sodium azide (32.3 g) was heated at 75° C. overnight. The mixture was allowed to cool to room temperature, diluted with water, and extracted twice with ethyl acetate. The combined extracts were washed sequentially with water and brine, and then dried (Na₂SO₄). Solvent was removed under reduced pressure to give a yellow oil that was purified by column chromatography on silica-gel [elution with ethyl acetate:hexanes (1:1)] to give the product azide as an off-white solid (49 g). The product could be further purified by trituration with ethyl acetate/hexanes.

¹H-NMR (DMSO-d₆) δ: 3.57-3.64 (dd, 1H); 3.70-3.77 (dd, 1H); 3.81-3.87 (dd, 1H); 4.06 (t, 1H); 4.78-4.84 (m, 1H); 7.05-7.09 (ddd, 1H); 7.45 (dd, 1H); 7.68-7.74 (dd, 1H).

### (5R)-3-(3-Fluoro-4-iodophenyl)-5-(1H-1,2,3-triazol-1-ylmethyl)-1,3-oxazolidin-2-one

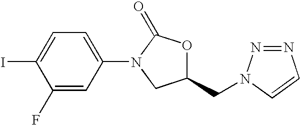 A stirred solution in dioxan (300 mL) of a mixture of the (5R)-5-azidomethyl-3-(3-fluoro-4-iodophenyl)-1,3-oxazolidin-2-one (30 g) and bicyclo[2.2.1]heptadiene (30 mL) was heated under reflux overnight. The mixture was allowed to cool to room temperature and then evaporated to dryness under reduced pressure to give a brown solid. The brown solid was purified by column chromatography on silica-gel [elution with a gradient from 98:2 to 95:5 methanol:chloroform] to give the product triazole as a pale yellow solid (20 g). The product could be further purified by trituration with dichloromethane/hexanes (1:1) to give an off-white solid.

¹H-NMR (DMSO-d₆) δ: 3.86-3.92 (dd, 1H); 4.23 (t, 1H); 4.83 (d, 2H); 5.11-5.19 (m, 1-1); 7.12-7.16 (dd, 1H); 7.47-7.51 (dd, 1H); 7.76 (s, 1H); 7.79-7.85 (dd, 1H); 8.16 (s, 1H).

### EXAMPLE 2

### (5R)-3-{2-Fluoro-4′-[5-(hydroxymethyl)-4,5-dihydroisoxazol-3-yl]-3′-methoxy-1,1′-biphenyl-4-yl}-5-(1H-1,2,3-triazol-1-ylmethyl)-1,3-oxazolidin-2-one

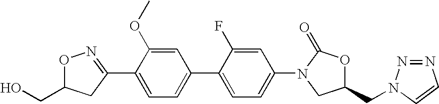 A solution of (5R)-3-{4′-[5-({[tert-Butyl(dimethyl)silyl]oxy}methyl)-4,5-dihydroisoxazol-3-yl]-2-fluoro-3′-methoxy-1,1′-biphenyl-4-yl}-5-(1H-1,2,3-triazol-1-ylmethyl)-1,3-oxazolidin-2-one (87 mg, 0.15 mmol) in tetrahydrofuran (2 mL) at room temperature was treated with a solution of tetrabutylammonium fluoride in tetrahydrofuran (1M; 0.18 mL, 0.18 mmol). The reaction mixture was stirred for 120 minutes then concentrated in vacuo. The resulting solid was dissolved in DMSO (2 mL) and purified by reverse phase HPLC (elution gradient 30% to 50% acetonitrile:water) to give a white solid that was washed with saturated sodium hydrogen carbonate solution and then dried to give the title compound 34 mg (49%).

MS (ESP+): (M+H)⁺468.00 for C₂H₂₃H N₅O₅ NMR (DMSO-d₆) δ: 3.26 (dd, 1H); 3.45 (dd, 1H); 3.55 (d, 21H); 3.81 (s, 3H); 3.96 (dd, 1H); 4.31 (t, 1H); 4.76 (m, 1H); 4.88 (d, 2H); 5.05 (s, 1H); 5.20 (m, 1H); 7.32 to 7.42 (m, 5H); 7.47 to 7.51 (dd, 1H); 7.80 (s, 1H); 8.21 (d, 1H).

### EXAMPLE 3

### (5R)-3-{4′-[5-({[tert-Butyl(dimethyl)silyl]oxy}methyl)-4,5-dihydroisoxazol-3-yl]-2,3′-difluoro-1,1′-biphenyl-4-yl}-5-(1H-1,2,3-triazol-1-ylmethyl)-1,3-oxazolidin-2-one

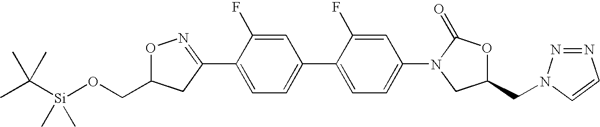 A mixture of (5R)-3-(3-Fluoro-4-iodophenyl)-5-(1H-1,2,3-triazol-1-ylmethyl)-1,3-oxazolidin-2-one (516 mg, 1.33 mmol), 5-({[tert-butyl(dimethyl)silyl]oxy}methyl)-3-[2-fluoro-4-(trimethylstannyl)phenyl]-4,5-dihydroisoxazole (758 mg, 1.6 mmol) and copper (1) iodide (104 mg, 0.53 mmol) in dry 1-methyl-2-pyrrolidinone (2 mL) was degassed and maintained under an atmosphere of argon. The mixture was treated with tetrakis(triphenylphosphine)palladium(0) (140 mg, 0.13 mmol) and the reaction mixture was stirred for 16 hours at 90° C. The reaction mixture was cooled and partitioned between aqueous potassium fluoride solution (100 mL, 2M) and ethyl acetate (100 mL). The ethyl acetate layer was separated, dried over magnesium sulphate, filtered, and the product was concentrated in vacuo onto Isolute HM-N (5 mL). The product was purified by chromatography (SiO₂ 20 g bond elut: elution gradient 0% to 5% methanol:dichloromethane) to give the title compound (499 mg 66%).

MS (ESP+): (M+H)⁺617.17 for C₂₈H₃₃F₂N₅O₄Si

NMR (DMSO-d₆) δ: 0.06 (s, 3H); 0.08 (s, 3H); 0.85 (s, 9H); 3.28 (m, 1H); 3.50 (dd, 1H); 3.75 (m, 2H); 3.98 (dd, 1H); 4.32 (t, 1H); 4.82 (m, 1H); 4.88 (d, 2H); 5.20 (m, 1H); 7.42 (m, 1H); 7.49 to 7.72 (m, 4H); 7.79 to 7.85 (m, 2H); 8.21 (s, 1H).

The intermediates for these compounds were prepared as follows

### 4-Bromo-2-fluorobenzaldehyde oxime

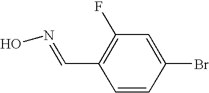 The title compound was prepared from 4-bromo-2-fluorobenzaldehyde by essentially the same method as that described in Example 1 for 4-bromo-2-methoxybenzaldehyde oxime NMR (DMSO-d₆) δ: 3.29 (s, 1H); 7.46 (d, 1H); 7.65 (d, 1H); 7.70 (t, 1H); 8.20 (s, 1H).

### [3-(4-Bromo-2-fluorophenyl-4,5-dihydroisoxazol-5-yl]methanol

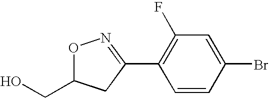

The title compound was prepared from 4-bromo-2-fluorobenzaldehyde oxime by essentially the same method as that described in Example 1 for [3-(4-bromo-2-methoxyphenyl)-4,5-dihydroisoxazol-5-yl]methanol.

MS (ESP+): (M+H)⁺274 & 276 for C₁₀H₉BrFNO₂

NMR (DMSO-d₆) δ: 3.25 (dd, 1H); 3.44 (dd, 1H); 3.50 to 3.61 (m, 2H); 4.74 (m, 1H); 5.01 (s, 1H); 7.52 (dd, 1H); 7.68 to 7.73 (m, 2H).

### 3-(4-Bromo-2-fluorophenyl-5-({[tert-butyl(dimethyl silyl]oxy}methyl)-4,5-dihydroisoxazole

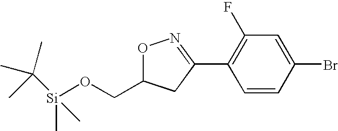 A stirred solution of [3-(4-bromo-2-fluorophenyl)-4,5-dihydroisoxazol-5-yl]methanol (1.388 g, 4.9 mmol) in a mixture of triethylamine (0.82 mL, 5.9 mmol) and dichloromethane (30 mL) was treated at 0° C. dropwise during 30 minutes with a solution of solution of tert-butyldimethylsilylchloride (1M; 5.4 mL) in dichloromethane and then with 4-dimethylaminopyridine (0.06 g, 0.5 mmol). The reaction mixture was stirred overnight at room temperature and then washed with water (100 mL). The dichloromethane layer was dried over magnesium sulfate and filtered and the product was concentrated in vacuo onto Isolute HM-N (10 mL). The product was purified by chromatography [SiO₂ 50 g bond elut; elution gradient 0% to 25% ethyl acetate:hexanes] to give the title compound (1.286 g 68%) as a solid.

MS (ESP+): (M+H)⁺387.90 & 389.9 for C₁₆H₂₃BrFNO₂Si

NMR (DMSO-d₆) δ: 0.05 (s, 3H); 0.06 (s, 3H); 0.83 (s, 9H); 3.25 (dd, 1H); 3.45 (dd, 1H); 3.66 to 3.80 (m, 2H); 4.79 (m, 1H); 7.52 (dd, 1H); 7.66 to 7.74 (m, 2H).

### 5-({[tert-Butyl(dimethyl)silyl]oxy}methyl)-3-[2-fluoro-4-(trimethylstannyl)phenyl]-4,5-dihydroisoxazole

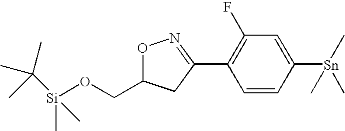 A stirred solution of 3-(4-bromo-2-fluorophenyl)-5-({[tert-butyl(dimethyl)silyl]oxy}methyl)-4,5-dihydroisoxazole (1.286 g, 3.31 mmol) in dry 1,4-dioxane (20 mL) was degassed and maintained under an atmosphere of argon. The mixture was treated with hexamethylditin (1.2 g, 3.64 mmol) and then with bis(triphenylphosphine)palladium(II) chloride (0.116 g, 0.17 mmol) and stirred at 90° C. for 90 minutes under an atmosphere of argon. The reaction mixture was cooled and solvent was removed in vacuo to give a crude product that was re-dissolved in ethyl acetate (100 mL), absorbed onto silica-gel (5 mL) and then purified by chromatography [SiO₂ 50 g bond elut: elution gradient 0% to 12.5% ethyl acetate:hexanes] to give the title compound (0.758 g 48%) as a solid. NMR (DMSO-d₆) δ: 0.05 (s, 3H); 0.07 (s, 3H); 0.32 (t, 9H); 0.84 (s, 9H); 3.23 (dd, 1H); 3.45 (dd, 1H); 3.73 (m, 2H); 4.77 (m, 1H); 7.37 to 7.43 (m, 2H); 7.67 (t, 1H).

### EXAMPLE 4

### (5R)-3-{2,3′-Difluoro-4′-[5-(hydroxymethyl)-4,5-dihydroisoxazol-3-yl]-1,1′-biphenyl-4-yl}-5-(1H-1,2,3-triazol-1-ylmethyl)-1,3-oxazolidin-2-one

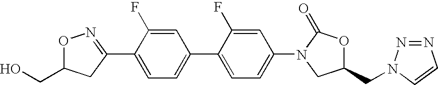 A stirred solution of (5R)-3-{4′-[5-({[tert-Butyl(dimethyl)silyl]oxy}methyl)-4,5-dihydroisoxazol-3-yl]-2,3′-difluoro-1,1′-biphenyl-4-yl}-5-(1H-1,2,3-triazol-1-ylmethyl)-1,3-oxazolidin-2-one (496 mg, 0.87 mmol) in dichloromethane (10 mL) was treated at room temperature with a solution of tetrabutylammonium fluoride in tetrahydrofuran (1M; 0.96 mL, 0.96 mmol) for 180 minutes. The product was then fractionated by chromatography [SiO_{2 20} g bond elut; elution gradient 0% to 6% methanol:dichloromethane] to give a crude product solution that was evaporated, treated with water (100 mL), and isolated by filtration to give the title compound (190 mg 40%).

MS (ESP+): (M+H)⁺455.98 for C₂₂H₁₉F₂N₅O₄

NMR (DMSO-d₆) δ: 3.27 (, 1H); 3.49 (dd, 1H); 3.55 (q, 2H); 3.98 (dd, 1H); 4.32 (t, 1H); 4.76 (m, 1H); 4.88 (d, 2H); 5.04 (t, 1H); 5.20 (m, 1H); 7.42 (dd, 1H); 7.49 to 7.61 (m, 3H); 7.69 (t, 1H); 7.79 (d, 1H); 7.84 (t, 1H); 8.21 (d, 1H).

1. A compound of the formula (I), or a pharmaceutically-acceptable salt, or an in-vivo-hydrolysable ester thereof, 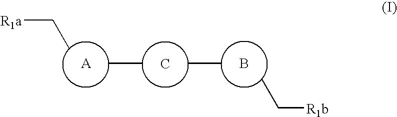
wherein in (I) C is a biaryl group C′-C″ 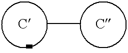
where C′ and C″ are independently aryl or heteroaryl rings such that the group C is represented by any one of the groups D to O below: 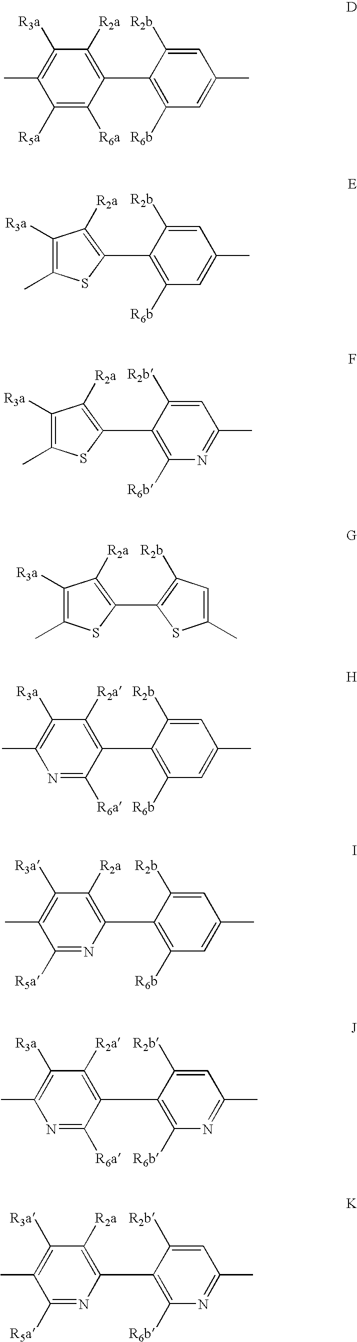 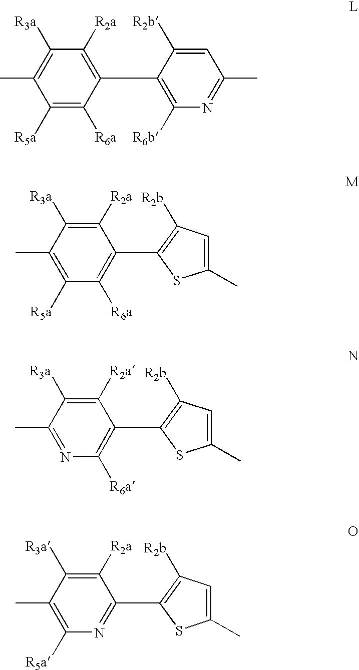
wherein the groups D to O are attached to rings A and B orientation [(A-C′) and (C″-B)] shown and
wherein A and B are independently selected from 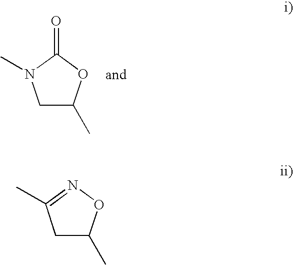
wherein i) and/or ii) are linked as shown in (I) via the 3-position to group C and substituted in the 5-position as shown in (I) by —CH₂—R₁a and —CH₂—R₁b;
R₂b and R₆b are independently selected from H, F, Cl, OMe, Me, Et and CF₃;
R₂b′ and R₆b′ are independently selected from H, OMe, Me, Et and CF₃;
R₂a and R₆a are independently selected from H, Br; F, Cl, OMe, SMe; Me, Et and CF₃;
R₂a′ and R₆a′ are independently selected from H, OMe, SMe; Me, Et and CF₃;
R₃a and R₅a are independently selected from H, (1-4C)alkyl, Br, F, Cl, OH, (1-4C)alkoxy, —S(O)ₙ(1-4C)alkyl (wherein n=0, 1, or 2), amino, (1-4C)alkylcarbonylamino, nitro, cyano, —CHO, —CO(1-4C) alkyl, —CONH₂ and —CONH(1-4C)alkyl;
R₃a′, R₅a′ are independently selected from H, (1-4C)alkyl, OH, (1-4C)alkoxy, (1-4C)alkylthio, amino, (1-4C)alkylcarbonylamino, nitro, cyano, —CHO, —CO(1-4C)alkyl, —CONH₂ and —CONH(1-4C)alkyl;
wherein any (1-4C)alkyl group may be optionally substituted with F, OH, (1-4C)alkoxy, —S(O)ₙ(1-4C)alkyl (wherein n=0, 1, or 2) or cyano;
wherein at least one of R₂a′, R₆a′, R₃a, R₅a, R₃a′, and R₅a′ is not H;
wherein when ring C′ is a pyridine ring (ie when group C is group H, I, J, K, N or O) the ring nitrogen may optionally be oxidised to an N-oxide;
R₁a and R₁b are independently selected from hydroxy, —OSi(tri-(1-6C)alkyl) (wherein the 3 (1-6C)alkyl groups are independently selected from all possible (1-6C)alkyl groups), —NR₅C(═W)R₄, —OC(═O)R₄, 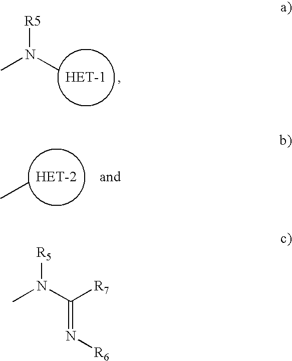
wherein W is O or S;
R₄ is hydrogen, amino, (1-8C)alkyl, —NHR₁₂, —N(R₁₂)(R₁₃), —OR₁₂ or —SR₁₂, (2-4C)alkenyl, (1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2; and wherein at each occurrence, alkyl, alkenyl, cycloalkyl cycloalkenyl in substituents in R₄ is optionally substituted with one, two, three or more F, Cl or CN;
R₅ is hydrogen, (3-6C)cycloalkyl, phenyloxycarbonyl, tert-butoxycarbonyl, fluorenyloxycarbonyl, benzyloxycarbonyl, (1-6C)alkyl (optionally substituted by cyano or (1-4C)alkoxycarbonyl), —CO₂R₈, —C(═O)R₈, —C(═O)SR₈, —C(═S)R₈, P(O)(OR₉)(OR₁₀) and —SO₂R₁₁, wherein R₈, R₉, R₁₀ and R₁₁ are as defined hereinbelow;
HET-1 is selected from HET-1A and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl;
HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
HET-2 is selected from HET-2A and HET-2B wherein
HET-2A is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from RT as hereinafter defined and/or on an available nitrogen atom, other than a N atom adjacent to the linking N atom, (provided that the ring is not thereby quaternised) by (1-4C)alkyl;
HET-2B is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from RT as hereinafter defined and/or on an available nitrogen atom, other than a N atom adjacent to the linking N atom, (provided that the ring is not thereby quaternised) by (1-4C)alkyl;
RT is selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or
(RTa2) (1-4C)alkylamino, di-(1-4C)alkylamino, and (2-4C)alkenylamino;
or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl;
or RT is selected from the group
(RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₆ is cyano, —COR₁₂, —COOR₁₂, —CONHR₁₂, —CON(R₁₂)(R₁₃), —SO₂R₁₂, —SO₂NHR₁₂, —SO₂N(R₁₂)(R₁₃) or NO₂, wherein R₁₂ and R₁₃ are as defined hereinbelow;
R₇ is hydrogen, amino, (1-8C)alkyl, —NHR₁₂, —N(R₁₂)(R₁₃), —OR₁₂ or —SR₁₂, (2-4C)alkenyl, (1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2;
R₈ is hydrogen, (3-6C)cycloalkyl, phenyl, benzyl, (1-5C)alkanoyl, (1-6C)alkyl (optionally substituted by substituents independently selected from (1-5C)alkoxycarbonyl, hydroxy, cyano, up to 3 halogen atoms and —NR₁₅R₁₆ (wherein R₁₅ and R₁₆ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₅)(R₁₆) group, R₁₅ and R₁₆ may additionally be taken together with the nitrogen atom to which they are attached to form a pyrrolidinyl, piperidinyl or morpholinyl ring);
R₉ and R₁₀ are independently selected from hydrogen and (1-4C)alkyl;
R₁₁ is (1-4C)alkyl or phenyl;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may additionally be taken together with the nitrogen atom to which they are attached to form a pyrrolidinyl, piperidinyl or morpholinyl ring which ring may be optionally substituted by a group selected from (1-4C)alkyl, (1-4C)cycloalkyl, (1-4C)acyl, —COO(1-4C)alkyl, S(O)ₙ(1-4C)alkyl (wherein n=1 or 2), —CS(1-4C)alkyl and —C(═S)O(1-4C)alkyl.

2. A compound of claim 1 wherein group C is represented by any one of groups D, E, H and I.

3. A compound of claim 2, wherein group C is represented by group D.

4. A compound of claim 2, wherein group C is represented by group H.

5. A compound of claim 4 wherein R₃a is methoxy, methyl or fluoro and R₅a is hydrogen.

6. A compound of claim 4 wherein R₃a is methoxy, methyl or fluoro and R₂a′ and R₆a′ are hydrogen; or R₃a and R₂a′ are hydrogen and R₆a′ is methyl or methoxy.

7. A compound of claim 1 wherein R₁a and R₁b are independently selected from —NHCO(1-4C)alkyl, —NHCO(1-4C)cycloalkyl, —NHCS(1-4C)alkyl, —N(R₅)-HET-1 and HET-2.

8. A compound of claim 1, wherein R₁a and R₁b are independently selected from hydroxy, —NHCO(1-4C)alkyl, and HET-2.

9. A compound of claim 1, wherein HET-2A is selected from the structures (Za) to (Zf) below: 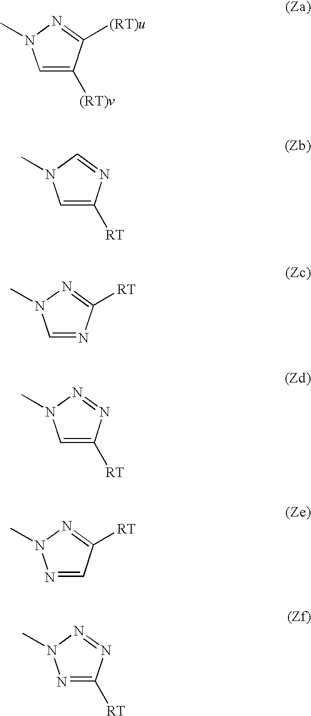
wherein u and v are independently 0 or 1.

10. A compound of claim 9 wherein RT is selected from
(a) hydrogen;
(b) halogen;
(c) cyano;
(d) (1-4C)alkyl;
(e) monosubstituted (1-4C)alkyl;
(f) disubstituted (1-4C)alkyl, and
trisubstituted (1-4C)alkyl.

11. A compound of claim 1 wherein at least one of A and B is an oxazolidinone.

12. A compound of claim 1 wherein both A and B are oxazolidinones.

13. A compound of claim 1 having formula (Ia).

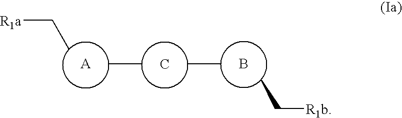

14. A pro-drug of a compound as claimed in any one of the preceding claims.

15. A method for producing an antibacterial effect in a warm blooded animal which comprises administering to said animal an effective amount of a compound of claim 1.

16. (canceled)

17. (canceled)

18. A pharmaceutical composition which comprises a compound of claim 1, and a pharmaceutically-acceptable diluent or carrier.

19. A process for the preparation of a compound of formula (I) as claimed in claim 1 or pharmaceutically acceptable salts or in-vivo hydrolysable esters thereof, which process comprises one of processes (a) to (h); and thereafter if necessary:
i) removing any protecting groups;
ii) forming a pro-drug (for example an in-vivo hydrolysable ester); and/or
iii) forming a pharmaceutically-acceptable salt;
wherein said processes (a) to (h) are:
(a) modifying a substituent in, or introducing a substituent into another compound of the invention by using standard chemistry;
(b) reaction of a molecule of a compound of formula (IIa) with a molecule of a compound of formula (IIb), wherein X and X′ are leaving groups useful in palladium coupling and are chosen such that an aryl-aryl, heteroaryl-aryl, or heteroaryl-heteroaryl bond replaces the aryl-X (or heteroaryl-X) and aryl-X′ (or heteroaryl-X′) bonds; 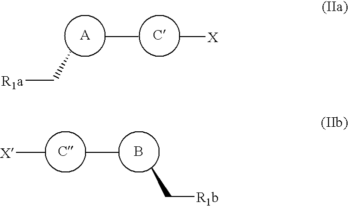
c) reaction of a (hetero)biaryl derivative (IIIa) or (IIIb) carbamate with an appropriately substituted oxirane to form an oxazolidinone ring at the undeveloped aryl position 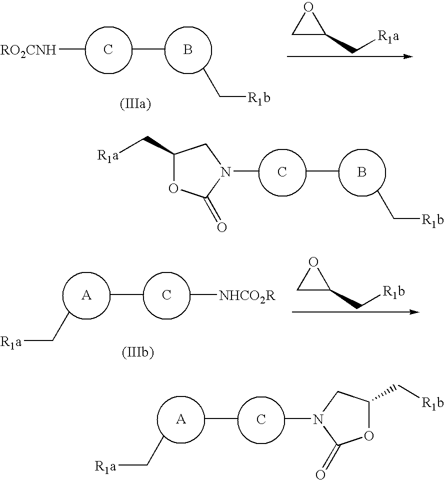
or by variations on this process in which the carbamate is replaced by an isocyanate or by an amine or/and in which the oxirane is replaced by an equivalent reagent X—CH₂CH(O-optionally protected)CH₂R₁a or X—CH₂CH(O-optionally protected)CH₂R₁b where X is a displaceable group;
d) reaction of a (hetero)biaryl derivative (IVa) or (IVb) to form an isoxazoline ring at the undeveloped aryl position; 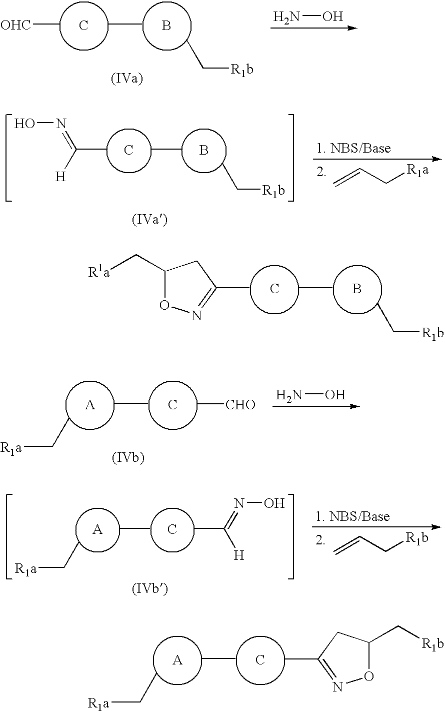
or by variations on this process in which the reactive intermediate (a nitrile oxide IVa′ or IVb″) is obtained other than by oxidation of an oxime (IVa′) or (IVb′); 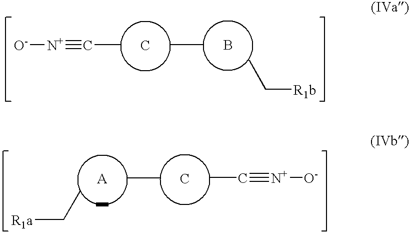
(e) for HET as optionally substituted 1,2,3-triazoles, compounds of the formula (I) by cycloaddition via the azide to acetylenes, or to acetylene equivalents such as optionally substituted cylcohexa-1,4-dienes or optionally substituted ethylenes bearing eliminatable substituents such as arylsulfonyl;
(f) for HET as 4-substituted 1,2,3-triazole compounds of formula (I) by reacting aminomethyloxazolidinones with 1,1-dihaloketone sulfonylhydrazones 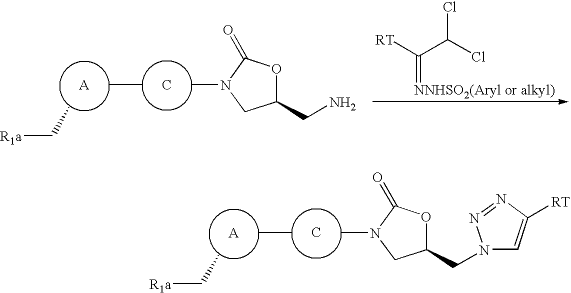
(g) for HET as 4-substituted 1,2,3-triazole compounds of formula (I), by reacting azidomethyl oxazolidinones with terminal alkynes using Cu(I) catalysis to give 4-substituted 1,2,3-triazoles
(h) for HET as 4-halogenated 1,2,3-triazole compounds of formula (I) may also be made by reacting azidomethyl oxazolidinones with halovinylsulfonyl chlorides at a temperature between 0° C. and 100° C. either neat or in an inert diluent, as shown below

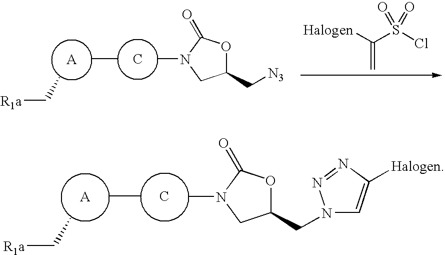

20. A pharmaceutical composition as claimed in claim 18, wherein said composition includes a vitamin.

21. A pharmaceutical composition as claimed in claim 20 wherein said vitamin is Vitamin B.

22. A pharmaceutical composition as claimed in claim 18, wherein said composition comprises a combination of a compound of the formula (I) and an antibacterial agent active against gram-positive bacteria.

23. A pharmaceutical composition as claimed in claim 18, wherein said composition comprises a combination of a compound of the formula (I) and an antibacterial agent active against gram-negative bacteria.
